SEQUENCE LISTING (1 sequence)

SEQ ID NO: 1 - protein, 53 aa, Pentadiplandra brazzeana
DKCKKVYENYPVSKCQLANQCNYDCKLDKHARSGECFYDEKRNLQCICDYCEY

(19) United States
(12) Patent Application Publication
(10) Pub. No.: US 2022/0361544 A1
(43) Pub. Date: Nov. 17, 2022

(54) Compositions Containing Brazzein

(71) Applicant: The Coca-Cola Company, Atlanta, GA (US)

(72) Inventor: Indra Prakash, Alpharetta, GA (US)

(21) Appl. No.: 17/600,745

(22) PCT Filed: Apr. 1, 2020
(86) PCT No.: PCT/US2020/026185

§ 371 Date: Oct. 1, 2021

Related U.S. Application Data
(60) Provisional application No. 62/827,487, filed on Apr. 1, 2019.

(51) Int. Cl. A23L 27/30; A23L 2/02; A23F 3/40; A23F 5/24

(52) U.S. Cl. CPC A23L 27/36; A23L 27/31; A23L 2/02; A23F 3/40; A23F 5/243

(57) ABSTRACT

Compositions comprising brazzein (or analogs thereof), including sweetener compositions, flavor modifying compositions, sweetened compositions, flavor modified compositions are disclosed. Also disclosed are methods of improving the temporal profile, flavor profile and/or taste profile of sweetenable compositions, such as beverages.

### CROSS-REFERENCE TO RELATED APPLICATION

This application claims priority to U.S. Provisional Application 62/827,487, filed Apr. 1, 2019, which is incorporated herein by reference in its entirety.

### FIELD OF THE INVENTION

Disclosed herein are sweetener compositions, flavor modifying compositions and methods of preparing and using the same. Also disclosed are consumables (e.g., beverages) containing such sweetener compositions and/or flavor modifying compositions.

### BACKGROUND OF THE INVENTION

Natural caloric sugars, such as sucrose, fructose and glucose, are utilized heavily in beverage, food, pharmaceutical, and oral hygienic/cosmetic industries due to their pleasant taste. Sucrose, in particular, imparts a taste preferred by consumers. Although sucrose provides superior sweetness characteristics, it is caloric. While calories are necessary for proper bodily functions, there is a preference among certain consumers for alternative non-caloric or low-caloric sweeteners with sugar-like taste. Non-caloric or low caloric sweeteners have been introduced to satisfy consumer demand. However, sweeteners within this group have been associated with undesirable taste characteristics. Specifically, non-caloric or low-caloric sweeteners exhibit a temporal profile, maximal response, flavor profile, mouth feel, and/or adaptation behavior that differs from sugar. When used in consumer products, delayed sweetness onset, lingering sweet aftertaste, bitter taste, metallic taste, astringent taste, cooling taste and/or licorice-like taste are often detected.

Accordingly, there remains a need to develop reduced or non-caloric sweeteners, and sweetened compositions, that are suitable for use in consumer products that also provide a temporal and flavor profile similar to that of sucrose.

### SUMMARY OF THE INVENTION

Disclosed herein are compositions comprising brazzein (or analogs thereof), as well as methods of making and using the same.

In a first embodiment, a sweetener composition or flavor modifying composition is disclosed comprising (i) brazzein (or an analog thereof) and (ii) at least one steviol glycoside or mogroside.

In a particular embodiment the steviol glycoside is selected from Rebaudioside M (“Reb M”), Rebaudioside A (“Reb A”) and A95. In a particular embodiment, the mogroside is siamenoside I.

In a second embodiment, a sweetener composition or flavor modifying composition is disclosed comprising (i) brazzein (or an analog thereof) and (ii) high fructose corn syrup (HFCS).

In a third embodiment, a consumable (e.g., a beverage) is provided comprising (i) brazzein (or an analog therefor) and (ii) at least one steviol glycoside or HFCS.

In a particular embodiment, the brazzein (or analog thereof) is present in an amount between about 1 ppm and about 50 ppm.

In a third embodiment, a method disclosed for imparting a more sugar-like temporal profile, flavor profile and/or taste profile to a consumable (e.g., a beverage or other ingestible consumable) by adding the sweetener composition or flavor modifying compositions disclosed herein to the consumable, thereby providing a consumable having a more sugar-like temporal profile, flavor profile and/or taste profile.

In one embodiment, the more sugar-like temporal profile is a reduced sweetness linger compared to a consumable to which the sweetener composition or flavor modifying composition disclosed herein had not been added.

In another embodiment, the more sugar-like flavor profile is an improved mouthfeel compared to a consumable to which the sweetener composition or flavor modifying composition disclosed herein had not been added. In a particular embodiment, the improved mouthfeel is increased body or fullness.

In a further embodiment, the more sugar-like taste profile reduced bitterness compared to a consumable to which the sweetener composition or flavor modifying composition disclosed herein had not been added.

The method can further include the addition of other sweeteners, additives, functional ingredients and combinations thereof.

The brazzein used in compositions disclosed herein (e.g., sweetener compositions, flavor modifying compositions, or consumables comprising the same) may be produced by any suitable means, such as extraction, chemical synthesis, in vivo or in vitro.

The one or more steviol glycosides can be used in any form. In one embodiment, the steviol glycoside is present in a Stevia extract, wherein the steviol glycoside constitutes from about 5% to about 100% of the Stevia extract by weight on a dry basis. In a further embodiment, the steviol glycoside is present in a mixture of steviol glycosides, wherein the steviol glycoside constitutes from about 5% to about 100% of the steviol glycoside mixture by weight on a dry basis.

The compositions disclosed herein can also contain one or more additional sweeteners, including, for example, natural sweeteners, high potency sweeteners, carbohydrate sweeteners, synthetic sweeteners and combinations thereof.

The compositions can also contain one or more additives including, for example, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof.

The compositions can also contain one or more functional ingredients, such as, for example, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

The disclosed consumables include, for example, pharmaceutical compositions, edible gel mixes and compositions, dental compositions, foodstuffs, beverages and beverage products.

In particular embodiments, beverages are disclosed containing the sweetener or flavor modifying compositions disclosed herein. The beverages contain a liquid matrix which forms the basis of the same, such as, for example, deionized water, distilled water, reverse osmosis water, carbon-treated water, purified water, demineralized water, phosphoric acid, phosphate buffer, citric acid, citrate buffer and carbon-treated water.

Low-calorie and zero-calorie beverages containing the sweetener compositions or flavor modifying compositions disclosed herein are also provided.

Tabletop sweetener compositions comprising the sweetener compositions or flavor and/or taste modifying compositions disclosed herein are also provided. The tabletop composition can further include at least one bulking agent, additive, anti-caking agent, functional ingredient and combinations thereof.

### DETAILED DESCRIPTION OF THE INVENTION

### Definitions

The term “analog,” as used herein, refers to molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. The modification may be, for example, the replacement, deletion or insertion of one or more amino acids compared to the wild-type polypeptide. In some embodiments, the analog has not more than 10, 9, 8 or 7 amino acids replaced, deleted or inserted compared to wild-type peptide or a portion thereof. In some embodiments, the analog has not more than 6 amino acids replaced, deleted or inserted compared to wild-type peptide or a portion thereof. In some embodiments, the analog has not more than 5 or 4 amino acids replaced, deleted or inserted compared to wild-type peptide or a portion thereof. In some embodiments, the analog has not more than 3, 2 or 1 amino acids replaced, deleted or inserted compared to wild-type peptide or a portion thereof. An analog may include an unnatural amino acid.

The term “astringency”, as used herein, refers to a perception puckering and dryness in the palate and is known to build in intensity and become increasingly difficult to clear from the mouth over repeated exposures. Astringency is a dry sensation experienced in the mouth and is commonly explained as arising from the loss of lubricity owing to the precipitation of proteins from the salivary film that coats and lubricates the oral cavity. Astringency is not confined to a particular region of the mouth but is a diffuse surface phenomenon, characterized by a loss of lubrication.

The term “bitter” or “bitter taste” as used herein refers to the perception or gustatory sensation resulting following the detection of a bitter tastant. The following attributes may contribute to bitter taste: astringent, bitter-astringent, metallic, bitter-metallic, as well as off-tastes, aftertastes and undesirable tastes including but not limited to freezer-burn and card-board taste, and/or any combinations of these. It is noted that, in the art, the term “off-taste” is often synonymous with “bitter taste.” Bitterness of substances can be compared with bitter taste threshold of quinine which is 1. (Guyton, Arthur C. (1991) Textbook of Medical Physiology. (8th ed). Philadelphia: W. B. Saunders; McLaughlin S., Margolskee R. F. (1994). “The Sense of Taste”. American Scientist. 82 (6): 538-545). Bitterness can be tested using a panel of subjects, as described herein, or in vitro, for example using a taste receptor cell line.

The term “consumable”, as used herein, refers to substances which are contacted with the mouth of man or animal, including substances which are taken into and subsequently ejected from the mouth and substances which are drunk, eaten, swallowed or otherwise ingested, and are safe for human or animal consumption when used in a generally acceptable range. Exemplary consumables include, but are not limited to, pharmaceutical compositions, edible gel mixes and compositions, dental compositions, foodstuffs (confections, condiments, chewing gum, cereal compositions baked goods dairy products, and tabletop sweetener compositions) beverages and beverage products. Consumables can be sweetened or unsweetened.

As used herein, the term “degrees Brix” refers to the sugar content of an aqueous solution. One degree Brix is 1 gram of sucrose in 100 grams of solution and represents the strength of the solution as percentage by weight (% w/w).

The term “expression”, as used herein, refers to transcription and/or translation processes occurring within a cell. The level of transcription of a nucleic acid sequence of interest in a cell can be determined on the basis of the amount of corresponding mRNA that is present in the cell. For example, mRNA transcribed from a sequence of interest can be quantitated by RT-PCR or by Northern hybridization (see Sambrook et al., 1999, supra). Polypeptides encoded by a nucleic acid of interest can be quantitated by various methods, e.g. by ELISA, by assaying for the biological activity of the polypeptide, or by employing assays that are independent of such activity, such as Western blotting or radioimmunoassay, using immunoglobulins that recognize and bind to the polypeptide (see Sambrook et al., 1999, supra).

The term “expression vector”, as used herein, refers to a nucleic acid providing all required elements for the expression of the comprised structural gene(s) in a host cell. Typically, an expression plasmid comprises a prokaryotic plasmid propagation unit, e.g. for E. coli, comprising an origin of replication, and a selectable marker, an eukaryotic selection marker, and one or more expression cassettes for the expression of the structural gene(s) of interest each comprising a promoter, a structural gene, and a transcription terminator including a polyadenylation signal. Gene expression is usually placed under the control of a promoter, and such a structural gene is said to be “operably linked to” the promoter. Similarly, a regulatory element and a core promoter are operably linked if the regulatory element modulates the activity of the core promoter.

The term “flavor” or “flavor characteristic”, as used herein, refers to the sensory perception of the components of taste, odor (aroma) and/or texture. The flavor profile of a composition is a quantitative profile of the relative intensities of all of the taste attributes exhibited. Such profiles often are plotted as histograms or radar plots. In certain embodiments, the flavor profile comprises one or more flavors which contribute to the sensory experience of a subject. In certain embodiments, modifying, changing or varying the combination of stimuli in a flavor profile can change the sensory experience of a subject.

The term “flavor modifying composition”, as used herein, refers to a composition that modulates, including enhancing, multiplying, potentiating, decreasing, suppressing, or inducing, the tastes, smells, flavors and/or textures of a natural or synthetic tastant, flavoring agent, taste profile, flavor profile and/or texture profile in a subject to whom it is administered.

The term “high intensity sweetener”, as used herein, refers to refers to any synthetic or semi-synthetic sweetener or sweetener found in nature that is many times sweeter than sucrose (e.g., 20 times and more, 30 times and more, 50 times and more or 100 times sweeter than sucrose).

The term “isosweet”, as used herein, refers to compositions that have equivalent sweetness. Generally, the sweetness of a given composition is typically measured with reference to a solution of sucrose. See “A Systematic Study of Concentration-Response Relationships of Sweeteners,” G. E. DuBois, D. E. Walters, S. S. Schiffman, Z. S. Warwick, B. J. Booth, S. D. Pecore, K. Gibes, B. T. Carr, and L. M. Brands, in Sweeteners: Discovery, Molecular Design and Chemoreception, D. E. Walters, F. T. Orthoefer, and G. E. DuBois, Eds., American Chemical Society, Washington, D.C. (1991), pp 261-276.

The term “mouthfeel”, as used herein, refers to the sensory and tactile properties of the consumable perceived when the composition contacts the mouth cavity and surfaces. The sensory and tactile properties include the texture, thickness, consistency and body.

The term “organoleptic” or “organoleptic characteristics”, as used herein, refers to the sensations perceived by the five senses while consuming a consumable (e.g., a food or beverage). Organoleptic quality thus involves taste and aroma as well as color and texture of the consumable. Organoleptic properties are subjective, and the impact vary from individual to individual. These sensory properties can be evaluated, e.g., by panels of trained or untrained individuals having the necessary sensory skills. Analytical methods may include, e.g., discrimination/difference and descriptive analysis. In certain embodiments, the organoleptic property or an improvement therein is experienced by a majority of individuals tested.

The term “ppm”, as used herein, means parts-per-million and is a weight relative parameter. A part-per-million is a microgram per gram, such that a component that is present at 10 ppm is present at 10 micrograms of the specific component per 1 gram of the aggregate mixture.

The term “purity”, as used herein, refers to material that is substantially or essentially free from components that normally accompany the compound as found in its native state. Purity and homogeneity are typically determined using analytical chemistry techniques. Particularly, in one embodiment, the compound is at least 85% pure, more preferably at least 90% pure, more preferably at least 95% pure, and most preferably at least 99% pure. In another embodiment, the compound is at least 90% pure, at least 91% pure, at least 92% pure, at least 93% pure, at least 95% pure, at least 95% pure, at least 96% pure, at least 97% pure, at least 98% pure or at least 99% pure.

As used herein, “sensory experience” refers to a subject's sensory perception of a taste, taste profile, flavor, flavor profile or texture profile.

The term “sour” or “sourness”, as used herein, refers to a taste that detects acidity. It is caused by a hydrogen atom, or ions. The more atoms present in a food, the more sour it will taste. The sourness of substances is rated relative to dilute hydrochloric acid, which has a sourness index of 1. By comparison, tartaric acid has a sourness index of 0.7, citric acid an index of 0.46, and carbonic acid an index of 0.06. A reduction in sour taste can be expressed as percentage sour taste inhibition. In one embodiment, the taste modifying compositions of the present invention reduce sour taste of a consumable (e.g., a beverage) by at least about 5%, at least about 10%, at least about 15%, at least about 20% or at least about 25% or more relative to a consumable that does not contain the taste modifying composition.

As used herein, the term “steviol glycoside(s)” refers to glycosides of steviol, including, but not limited to, naturally occurring steviol glycosides, e.g. Rebaudioside A (Reb A), Rebaudioside B (Reb B), Rebaudioside C (Reb C), Rebaudioside D (Reb D), Rebaudioside E (Reb E), Rebaudioside F (Reb F), Rebaudioside G (Reb G), Rebaudioside H (Reb H), Rebaudioside I (Reb I), Rebaudioside J (Reb J), Rebaudioside K (Reb K), Rebaudioside L (Reb L), Rebaudioside M (Reb M), Rebaudioside N (Reb N), Rebaudioside O (Reb O), Rebaudioside Q (Reb Q), Rebaudioside R (Reb R), Rebaudioside S (Reb S), Rebaudioside T (Reb T), Rebaudioside U (Reb U), Rebaudioside V (Reb V), Rebaudioside W (Reb W), Rebaudioside Y (Reb Y), Stevioside, Steviolbioside, Dulcoside A and Rubusoside, etc. or synthetic or biosynthetic steviol glycosides, e.g. enzymatically glycosylated steviol glycosides, steviol glycoside products from bioconversion of steviol glycosides by biocatalysts, steviol glycosides from fermentation of recombinant microbial host capable of de novo synthesis of steviol glycosides, and combinations thereof. In a particular embodiment, the steviol glycoside is a rebaudioside analog. Steviol glycosides range in sweetness from 40 to 300 times sweeter than sucrose and are also characterized as being heat-stable, pH-stable, and non-fermentable.

The term “sucrose equivalence”, as used herein, refers to the sweetness of a non-sucrose composition against a sucrose reference. Typically, taste panelists are trained to detect sweetness of reference sucrose solutions containing between 1-15% sucrose (w/v). Other non-sucrose sweeteners are then tasted at a series of dilutions to determine the concentration of the non-sucrose sweetener that is as sweet (i.e. isosweet) to a given percent sucrose reference.

The term “sugar-like characteristic” refers to any characteristic similar to that of sucrose and include, but are not limited to, maximal response, flavor profile, taste profile, temporal profile, adaptation behavior, mouthfeel, concentration/response function, tastant/and flavor/sweet taste interactions, spatial pattern selectivity, and temperature effects. These characteristics are dimensions in which the taste of sucrose is different from the tastes of other compounds. The term “sweetener composition”, as used herein, mean compositions that contain at least one sweet component in combination with at least one other substance, such as, for example, another sweetener or an additive.

The term “sweetenable compositions”, as used herein, mean substances which are contacted with the mouth of man or animal, including substances which are taken into and subsequently ejected from the mouth and substances which are drunk, eaten, swallowed or otherwise ingested, and are safe for human or animal consumption when used in a generally acceptable range. For example, a beverage with no sweetener component is a type of sweetenable composition. A sweetener composition comprising Reb X and erythritol can be added to the un-sweetened beverage, thereby providing a sweetened beverage. In another example, a beverage containing Reb M or Reb A is a type of sweetenable composition. A sweetener composition comprising brazzein can be added to this Reb M or Reb A-containing beverage, thereby providing a sweetened beverage.

The term “sweetened compositions”, as used herein, mean substances that contain both a sweetenable composition and a sweetener or sweetener composition.

The term “sweetness recognition threshold concentration,” as generally used herein, is the lowest known concentration of a sweet compound that is perceivable by the human sense of taste, typically around 1.0% sucrose equivalence (1.0% SE). The sweetness recognition threshold concentration can be easily determined by taste testing increasing concentrations of a given enhancer until greater than 1.0% sucrose equivalence in a given beverage matrix is detected. The concentration that provides about 1.0% sucrose equivalence is considered the sweetness recognition threshold.

The term “synergism”, as used herein, refers to a condition where a combination of substances or compounds generates a higher activity (chemical and/or biological) than the sum of their individual activities. In one embodiment, the activity is sweetness.

The term “taste”, as used herein, refers to a sensation or perception caused by activation or inhibition of receptor cells in a subject's taste buds. Taste buds are able to distinguish between different tastes through detecting interaction with different molecules or ions. The tastes are considered to include sweet, sour, salt, bitter (the so-called “basic tastes”) as well as kokumi and umami.

As used herein, “texture profile” or “mouthfeel” refers to a composition's physical and chemical interaction in the mouth. The texture profile of a composition can include one or more texture, such as, for example, but not limited to, mouthwatering, lubricating, slippery, astringency, hardness, cohesiveness, viscosity, elasticity, adhesiveness, brittleness, chewiness, gumminess, moisture content, grittiness, smoothness, oiliness and greasiness. In certain embodiments, the texture profile can comprise one or more texture characteristic in the same or different intensities.

Sweetener Compositions/Flavor Modifying Compositions

Disclosed herein are sweetener compositions and flavor modifying compositions, in each case, containing brazzein (or analogs thereof). In certain embodiments, the sweetener compositions and flavor modifying compositions change (e.g., improve) one or more sensory experiences of a subject who consumes the same.

A flavor modifying composition may modify (e.g., enhance, inhibit or change) a taste, aroma and/or texture of a given composition, e.g., a consumable. In a particular embodiment, the flavor modifying composition modifies (e.g., enhances, inhibits or changes) a particular taste(s). In another embodiment, the flavor modifying composition modifies (e.g., enhances, inhibits or changes) a given texture. In certain embodiments, the flavor modifying composition modifies (e.g., enhances, inhibits or changes) both a given taste(s) and texture.

A flavor modifying composition may be sweetened or unsweetened. Therefore, in some embodiments, the addition of a flavor modifying composition may serve both to add flavor modifiers and may further provide sweetness to a composition selected for taste adjustment. The addition of a sweetened flavor modifying composition may be used in addition to or alternatively to addition of another sweetening composition.

The sweetener compositions and flavor modifying compositions disclosed herein contain brazzein, variants or analogs thereof. In certain embodiments, brazzein (or analog thereof) is the only sweet tasting component in the sweetener composition or flavor modifying composition. In certain embodiments, the sweetener composition or flavor modifying composition further comprises one or more additional sweet tasting components. In a particular embodiment, the one or more sweet tasting components include steviol glycosides (e.g., Reb M, Reb A) and HFCS.

Brazzein is a small, sweet-tasting protein originally isolated from the fruit of West African plant, Pentadiplandra brazzeana Baillon. (Ming D et al., FEBS Lett. (1994) 355:106-108). It is a monomer protein with a molecular weight of 6.5 kd. As a member of the Csβα fold family, it contains four disulfide bonds that lend a high degree of thermal and pH stability to its structure. Specifically, the sweet taste of brazzein remains after incubation at 98° C. for 2 h and at 80° C. for 4.5 h in the pH range of 2.5-8. It is also water soluble (>50 mg/mL). It is a highly soluble protein (more than 50 g/L) with an isoelectric point of 5.4

At least three forms of the protein are known, although only two forms are present in ripe fruit. The major form (pGlu-brazzein) (about 80%) contains a pyroglutamate (pGlu) residue at its N-terminus, and the minor form (des-pGlu1-brazzein, commonly referred to as “brazzein”) (about 20%) lacks that residue.

The 53-amino acid sequence of wild-type brazzein, minor form, is shown in SEQ ID NO: 1. The compositions disclosed herein may contain the wild-type brazzein or a naturally occurring variant or recombinant analog thereof.

The minor form has nearly twice the sweetness of the major form. Specifically, brazzein has a potency between about 500 and 2000 that of sucrose. Its taste is more similar to sucrose than that of thaumatin, another sweet tasting protein. In a particular embodiment, the sweetener composition or flavor modifying composition disclosed herein comprises the minor form of wild-type brazzein.

Brazzein is believed to participate in a multipoint binding interaction with the human sweet taste receptor, a heterodimeric G-protein-coupled receptor composed of subunits Taste type 1 Receptor 2 (T1R2) and Taste type 1 Receptor 3 (T1R3). (Assadi-Porter F M, et al., J Mol. Bio. (2010) 14; 398(4):584-99). The brazzein binding site is distinct from the sucrose binding site.

In a particular embodiment, the sweetener composition and flavor modifying compositions disclosed herein comprise a brazzein analog. In one embodiment, the brazzein analog differs from wild-type brazzein at least one amino acid position and more particularly, at one, two, three or more amino acid positions. In a particular embodiment, the wild-type amino acid sequence of the brazzein analog is conserved at site 1 (Loop residue R43), site 2 (N- and C-terminal regions/residue E36 and Loop 33), or site 3 (Loop residues 9-19). In a particular embodiment, the brazzein analog is selected from the group including Asp40Ala, Asp40Ly, Glu41Ala, Lys42Ala. Asp50Lys, Tyr54Trp, Asp29Ala/Glu41Lys, Asp29Asn/Glu41Lys, Asp29Lys/Glu41Lys.

In one embodiment, the sweetener composition and flavor modifying composition disclosed herein comprises a brazzein analog having a sweetness equal to or greater than wild-type brazzein.

In one embodiment, the sweetener composition and flavor modifying composition disclosed herein comprise a brazzein analog having a stability equal to or greater than wild-type brazzein.

Brazzein (or analogs thereof) suitable for use in compositions disclosed herein (e.g., sweetener compositions, flavor and/or taste modifying compositions, consumables) may be produced in any suitable manner. Representative methods of production include extraction, chemical synthesis (i.e., solid state synthesis) or recombinant production (i.e., in vivo production or in vitro production).

In one embodiment, brazzein used in the compositions disclosed herein is isolated from edible fruit of Pentadiplandra brazzeana Baillon, for example as described in WO 97/94/19467. The content of brazzein in the pulp of the fruit between the pericarp and the seeds is between about approximately 0.2-0.05% by weight.

In another embodiment, brazzein (or analog thereof) used in compositions disclosed herein is produced in vivo. In one embodiment, the nucleic acid coding sequence for the minor form of brazzein isolated from Pentadiplandra brazzeana fruit (or analog thereof), optionally optimized, is introduced into a suitable vector, which is then cloned into a host cell in an appropriate growth system/environment—resulting in expression of the protein in recombinant fashion. In one embodiment, the host cell is a prokaryotic cell or eukaryotic cell.

In certain embodiments, brazzein (or an analog thereof) is produced in a cell-free system, i.e., in vitro synthesis. A cell-free system is a system capable of translating a polynucleotide into a peptide, polypeptide, and/or protein that does not take place in an intact cell. Cell-free systems that can be used to produce brazzein for use in the compositions described herein include, but are not limited to, protein expression components from eukaryotic, prokaryotic, and/or viral sources. For example, cell-free systems as used herein can include mammalian and/or bacterial protein expression systems derived from mammalian and/or bacterial lysates.

In certain embodiments, recombinant brazzein is produced by a transgenic mammal, i.e., in milk.

In certain embodiments, brazzein (or analog thereof) may be expressed as a fusion protein. In a particular embodiment, the fusion protein comprises brazzein and a fusion tag, such as an affinity tag (e.g., a His tag) or a solubility-enhancing tag (e.g., a GST tag).

The expression of brazzein (or analog thereof) may be stable or transient. In a stable expression system, the exogenous DNA is integrated into the chromosomes, or as an episome a separate piece of nuclear DNA) and is passed on to future generations of the host cell.

In a cell-based system, the first step in the protein purification process to extract the protein from the cells by lysing or breaking them open. Any suitable cell lysis method may be used, for example, mechanical disruption, chemical breakdown, freeze-thaw cycles or enzymatic digestion. The protein can then by purified by any suitable protein purification method for example, affinity chromatography, ion exchange chromatography, filtration, electrophoresis, hydrophobic interaction chromatography, gel filtration chromatography, reverse phase chromatography, concanavalin A chromatography, chromatofocusing and differential precipitation or solubilization.

The yield of in vivo production of brazzein (or analog thereof) may vary. In a particular embodiment, brazzein represents at least about 1% of total cellular proteins. In a particular embodiment, brazzein represents between about 1% and about 5% of total cellular proteins. In another embodiment, brazzein represents between about 5% and about 10% or total cellular proteins. In a further embodiment, brazzein represents between 10% and about 20% of total cellular proteins. In certain embodiments, brazzein represents more than 20% of total cellular proteins.

In another particular embodiment, brazzein is purified to provide a yield between about 1 mg/mL and about 200 mg/mL, more particularly, between about 20 mg/mL and about 180 mg/mL, between about 40 mg/mL and about 160 mg/mL, between about 60 mg/mL and about 140 mg/mL or about 80 mg/mL and about 120 mg/mL. In one embodiment, the brazzein is purified to provide a yield of about 25 mg/mL, about 50 mg/mL, about 75 mg/mL, about 100 mg/mL, about 125 mg/mL, about 150 mg/mL. about 175 mg/mL, or about 200 mg/L or more. Optionally, the brazzein is produced as a fusion protein further comprising a tag and the yields described above reflect both purification and removal of the tag.

In a particular embodiment, the brazzein (or analog thereof) is substantially pure. In one embodiment, brazzein (or analog thereof) is at least about 80% pure, at least about 85% pure, at least about 90% pure, at least about 95% pure or at least about 99% pure. In another embodiment, brazzein (or analog thereof) is about 90%, about 91%, about 92%, about 93%, about 94%, about 95%, about 96%, about 97%, about 98% or about 99% pure.

In a particular embodiment, the brazzein (or analog thereof) used in the compositions disclosed herein is produced in yeast expression system (i.e., yeast-derived brazzein or analog thereof), for example, in the genera Kluyveromyces (e.g., K. lactis), Lactococcus (e.g., L. lactis), Lactobacillus, Saccharomyces (e.g., S. cerevisiae) Pischia (e.g., P. pastoris), Hansenula (e.g., H. polymorpha) or Yarrowia (e.g., Y. lipolytica).

In another particular embodiment, the brazzein (or analog thereof) used in the compositions disclosed herein is produced in a bacterial expression system (i.e., bacteria-derived brazzein or analog thereof), for example, in Escherichia coli or Bacillus subtilis. In one embodiment, the brazzein (or analog therefore) is not produced in E. coli.

In a further particular embodiment, the brazzein (or analog thereof) used in the compositions disclosed herein is produced in an insect expression system (i.e., insect-derived brazzein or analog thereof), for example, in baculovirus infected or non-lytic insect cells (e.g., sf9, Sf21).

In another embodiment, the brazzein (or analog thereof) used in compositions disclosed herein is produced in a fungal expression system (i.e., fungi-derived brazzein or analog thereof), for example in the genera Chrysosporium, Thielavia, Talaromyces, Thermomyces or Thermoascus.

In a further embodiment, brazzein (or analog thereof) is produced in an algal expression system (i.e., algae-derived brazzein or analog thereof).

In another embodiment, brazzein (or analog thereof) is produced in a plant expression system (i.e., plant-derived brazzein or analog thereof), for example, in maize, tobacco, potatoes, strawberries or sugarcane. In one embodiment, the plant expression system is a plant cell culture expression system.

In a particular embodiment, brazzein (or analog thereof) is produced in maize, and more particularly, the seeds of maize. According to this embodiment, brazzein (or analog thereof) may be utilized as brazzein-containing germ flour.

In yet another embodiment, brazzein (or analog thereof) is produced in a mammalian expression system (i.e., mammalian-derived brazzein or analog thereof), for example in Chinese hamster ovary (CHO) cells, human embryonic kidney (HEK), COS and baby hamster kidney (BHK) cells.

Alternatively, the brazzein (or analog thereof) used in the compositions disclosed herein may be produced in vitro using a cell-free expression system, such as an E. coli S30 extract.

The amount of brazzein (or analog thereof) in the sweetener composition and flavor modifying compositions disclosed herein may vary. In one embodiment, the brazzein (or analog thereof) is present above its sweetness threshold concentration.

In one embodiment, brazzein (or analog thereof) is present in the sweetener composition or flavor modifying composition in any amount to impart the desired sweetness when the sweetener composition or flavor modifying composition is added to a consumable (e.g., beverage), either alone or in combination with one or more additional sweet tasting components (e.g., steviol glycosides, HFCS) present in the sweetener composition or flavor modifying composition, i.e., before such compositions are added to the consumable.

In a particular embodiment, the desired sweetness of the consumable is isosweet to a sucrose-sweetened consumable having a sweetness from at least about 8 degrees Brix, such as, for example, about 9 degrees Brix, about 10 degrees Brix, about 11 degrees Brix, about 12 degrees Brix, about 13 degrees Brix, about 14 degrees Brix or about 15 degrees Brix.

In another embodiment, the desired sweetness of the consumable is isosweet to a sucrose-sweetened consumable having a sweetness from about 10 degrees Brix to about 15 degrees Brix, such as, for example, from about 10 degrees Brix to about 14 degrees Brix, from about 10 degrees Brix to about 13 degrees Brix, from about 10 degrees Brix to about 12 degrees Brix, from about 10 degrees Brix to about 11 degrees Brix, from about 11 degrees Brix to about 15 degrees Brix, from about 11 degrees Brix to about 14 degrees Brix, from about 11 degrees Brix to about 13 degrees Brix, from about 11 degrees Brix to about 12 degrees Brix, from about 12 degrees Brix to about 15 degrees Brix, from about 12 degrees Brix to about 14 degrees Brix, from about 12 degrees Brix to about 13 degrees Brix, from about 13 degrees Brix to about 15 degrees Brix, from about 13 degrees Brix to about 14 degrees Brix and from about 14 degrees Brix to about 15 degrees Brix.

In one embodiment, the brazzein (or analog thereof) is present in the sweetener composition or flavor modifying composition in an amount that enhances the sweetness of the consumable to which it is added by about 1.0% (w/v) sucrose equivalence (SE) or greater, either alone or in combination with one or more additional sweet tasting component (e.g. steviol glycosides, HFCS) present in the sweetener composition or flavor modifying composition, i.e., before such compositions are added to the consumable.

In a particular embodiment, the brazzein (or analog thereof) is present in the sweetener composition in an amount that enhances the sweetness of the consumable to which it is added from about 1.0% to about 3.0% (w/v) sucrose equivalence (SE), such as, for example, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9% or about 3.0% sucrose equivalence, either alone or in combination with one or more additional sweet tasting components (e.g., steviol glycosides, HFCS) present in the sweetener composition or flavor modifying composition, i.e., before such compositions are added to the consumable.

In another particular embodiment, the brazzein (or analog thereof) is present in the sweetener composition or flavor modifying composition in an amount that enhances the sweetness of the consumable to which it is added by about 3.0% to about 5% (w/v) sucrose equivalence (SE), for example, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9% or about 5.0%, either alone or in combination with one or more additional sweet tasting components (e.g., steviol glycosides, HFCS) present in the sweetener composition or flavor modifying composition, i.e., before such compositions are added to the consumable.

The sweetness of a given composition is typically measured with reference to a solution of sucrose. See generally “A Systematic Study of Concentration-Response Relationships of Sweeteners,” G. E. DuBois, D. E. Walters, S. S. Schiffman, Z. S. Warwick, B. J. Booth, S. D. Pecore, K. Gibes, B. T. Carr, and L. M. Brands, in Sweeteners: Discovery, Molecular Design and Chemoreception, D. E. Walters, F. T. Orthoefer, and G. E. DuBois, Eds., American Chemical Society, Washington, D.C. (1991), pp 261-276.

The amount of sucrose in a reference solution may be described in degrees Brix (° Bx). One degrees Brix is 1 gram of sucrose in 100 grams of solution and represents the strength of the solution as percentage by weight (% w/w) (strictly speaking, by mass).

In one embodiment, a sweetener composition is provided that contains brazzein (or analog thereof) in an amount effective to provide sweetness equivalent from about 1 to about 12 degrees Brix of sugar when added to a consumable, such as, for example, from about 2 to about 9 degrees Brix, from about 3 to about 8 degrees Brix, from about 4 to about 7 degrees Brix, or about 5 degrees Brix, either alone or together with one or more sweet tasting components (e.g., steviol glycosides, HFCS) present in the sweetener composition or flavor modifying composition or consumable. In another embodiment, brazzein (or analog thereof) is present in an amount effective to provide sweetness equivalent to about 10 degrees Brix when added to a sweetenable composition, either alone or in combination with one or more sweet tasting component (e.g., steviol glycosides, HFCS) present in the sweetener composition, flavor modifying composition or the consumable to which it is added.

The sweetness of a non-sucrose sweetener can also be measured against a sucrose reference by determining the non-sucrose sweetener's sucrose equivalence. Typically, taste panelists are trained to detect sweetness of reference sucrose solutions containing between 1-15% sucrose (w/v). Other non-sucrose sweeteners are then tasted at a series of dilutions to determine the concentration of the non-sucrose sweetener that is as sweet as a given percent sucrose reference. For example, if a 1% solution of a sweetener is as sweet as a 10% sucrose solution, then the sweetener is said to be 10 times as potent as sucrose.

In one embodiment, the amount of brazzein (or analog thereof) present in the sweetener composition or flavor modifying composition disclosed herein is any amount that contributes to one or more improved organoleptic properties of the consumable (e.g., beverage) to which the sweetener composition or taste modifying composition is added. In a particular embodiment, the improved organoleptic property is associated with taste. In one embodiment, improving one or more organoleptic property results in the improvement of the taste profile. The overall taste profile of a composition is an interplay of several different tastes, such as sweetness, sourness and the like.

Examples of improved organoleptic properties can include, for example, a reduction in bitterness, a reduction in astringent and liquorice notes, slower onset of sweetness, a reduction in lingering sweetness, a reduction in lingering bitterness, a reduction in bitter aftertaste, a reduction in metallic aftertaste, a reduction in chemical and synthetic aftertaste, and a combination thereof. In a particular embodiment, the term “improved organoleptic properties” means that the sweetened or taste modified composition (e.g., a beverage) will have one or more improved organoleptic properties for the majority of users. The improvement may be expressed qualitatively or quantitatively, e.g. as a percentage improvement.

The improved organoleptic property may be measured by or using technical means such as a taste sensing system (TSS), a term referring to analytical sensory array units (e.g., electrochemical, gravimetrical, optic or biosensors) which can detect specific substances. Sliwi'nska, M et al. J. Agric. Food Chem. (2014), 62, 1423-1448.

In a particular embodiment, the brazzein (or analog thereof) is present in the sweetener composition or flavor modifying composition in any amount that reduces, suppresses or masks the bitterness of a consumable (e.g., a beverage) to which the sweetener or flavor modifying composition is added, either alone or together with one or more sweet tasting components (e.g., steviol glycosides, HFCS) in the sweetener composition or flavor modifying composition, i.e., before it is added to the consumable. The comparison is made to a consumable to which the sweetener or flavor modifying composition has not been added.

In a particular embodiment, the brazzein (or analog thereof) is present in the sweetener composition or flavor modifying composition in an amount that reduces the bitterness of consumable (e.g., beverage) to which it is added by at least about 5%, at least about 10%, at least about 15%, at least about 20% or at least about 25% or more, either alone or in combination with one or more sweet taste components (e.g., steviol glycosides, HFCS) in the sweetener composition or flavor modifying composition, i.e., before it is added to the consumable. In one embodiment, the reduction in bitterness is experienced by a majority of subjects. The comparison is made to a consumable to which the sweetener or flavor modifying composition has not been added.

In a particular embodiment, the brazzein (or analog thereof) is present in the sweetener composition or flavor modifying composition in any amount that reduces the bitter aftertaste of a consumable (e.g., a beverage) to which the sweetener or flavor modifying composition is added, either alone or in combination with one or more sweet taste components (e.g., steviol glycosides, HFCS) in the sweetener composition or flavor modifying composition, i.e., before it is added to the consumable. In a particular embodiment, the brazzein (or analog thereof) is present in the sweetener composition or flavor modifying composition in an amount that reduces bitter aftertaste of consumable (e.g., beverage) to which it is added by at least about 5%, at least about 10%, at least about 15%, at least about 20% or at least about 25% or more. In one embodiment, the reduction in bitter aftertaste is experienced by a majority of subjects. The comparison is made to a consumable to which the sweetener or flavor modifying composition has not been added.

In another embodiment, the brazzein (or analog thereof) is present in the sweetener composition or taste modifying composition in any amount reduces the sweetness linger of a consumable (e.g., a beverage) to which the sweetener or taste modifying composition is added. Sucrose exhibits a sweet taste in which the maximal response is perceived quickly and where perceived sweetness disappears relatively quickly on swallowing a food or beverage. In contrast, the sweet tastes of essentially all high-potency sweeteners reach their maximal responses somewhat more slowly and they then decline in intensity more slowly than is the case for sucrose. This decline in sweetness is often referred to as “sweetness linger” and is a major limitation for high-potency sweeteners including NHPSs. Slow onset of sweetness also can be a problem. In general, however, sweetness linger is a more significant problem. And so, preferred embodiments of this invention exhibit significant reductions in sweetness linger.

In a particular embodiment, the brazzein (or analog thereof) is present in the sweetener composition or flavor modifying compositions in an amount that reduces the sweetness linger of the consumable (e.g., beverage) to which it is added by at least about 5%, at least about 10%, at least about 15%, at least about 20% or at least about 25% or more, either alone or in combination with one or more sweet tasting components (e.g., steviol glycosides, HFCS) in the sweetener composition or flavor modifying composition before it is added to the consumable. In one embodiment, the majority of subjects perceive the reduction in sweetness linger. In a particular embodiment, the comparison is made to a consumable to which the sweetener composition or flavor composition has not been added.

In certain embodiments, the sweetener composition or flavor and/or taste modifying compositions contain one or more additional sweeteners. In one embodiment, the additional sweetener is present above its sweetness threshold concentration. In certain embodiments, the sweetener composition containing brazzein and the one or more additional sweeteners synergistically enhance the sweetness of the consumable to which the sweetener composition is added. In one embodiment, the sweetness of the consumable is enhanced in a manner that would be unexpected to one of skill in the art.

The additional sweetener can be any type of sweetener, for example, a natural, non-natural, or synthetic sweetener.

As used herein the term “high-intensity sweetener,” refers to any synthetic or semi-synthetic sweetener or sweetener found in nature. High-intensity sweeteners are compounds or mixtures of compounds which are sweeter than sucrose. High-intensity sweeteners are typically many times (e.g., 20 times and more, 30 times and more, 50 times and more or 100 times or more sweeter than sucrose).

In at least one embodiment, the at least one additional sweetener is chosen from natural sweeteners other than Stevia sweeteners. In another embodiment, the at least one additional sweetener is chosen from synthetic high potency sweeteners (SHPS).

In a particular embodiment, the one or more additional sweetener may be a natural high potency sweetener (NHPS). Suitable natural high potency sweeteners include, but are not limited to, rebaudioside A, rebaudioside B, rebaudioside C, rebaudioside D, rebaudioside E, rebaudioside F, dulcoside A, dulcoside B, rubusoside, Stevia, stevioside, mogroside IV, mogroside V, Luo Han Guo sweetener, siamenoside, monatin and its salts (monatin SS, RR, RS, SR), curculin, glycyrrhizic acid and its salts, thaumatin, monellin, mabinlin, brazzein, hernandulcin, phyllodulcin, glycyphyllin, phloridzin, trilobatin, baiyunoside, osladin, polypodoside A, pterocaryoside A, pterocaryoside B, mukurozioside, phlomisoside I, periandrin I, abrusoside A, steviolbioside and cyclocarioside I. The natural high potency sweetener can be provided as a pure compound or, alternatively, as part of an extract. For example, rebaudioside A can be provided as a sole compound or as part of a Stevia extract.

In one embodiment, the one or more additional sweeteners is selected from the group consisting of rebaudioside M, rebaudioside A, siamenoside I and mogroside V.

In a particular embodiment, the sweetener composition and/or flavor modifying composition of the present invention comprises brazzein (or analog thereof) and siamenoside I.

In another particular embodiment, the sweetener composition and/or flavor modifying composition of the present invention comprises brazzein (or analog thereof) and mogroside V.

In another embodiment, the one or more additional sweeteners is selected from the group consisting of rebaudioside D, rebaudioside N, rebaudioside O, rebaudioside E, steviolmonoside, steviolbioside, rubusoside, dulcoside B, dulcoside A, rebaudioside B, rebaudioside G, stevioside, rebaudioside C, rebaudioside F, rebaudioside I, rebaudioside H, rebaudioside L, rebaudioside K, rebaudioside J, rebaudioside M2, rebaudioside D2, rebaudioside S, rebaudioside T, rebaudioside U, rebaudioside V, rebaudioside W, rebaudioside Z1, rebaudioside Z2, rebaudioside IX, enzymatically glucosylated steviol glycosides and combinations thereof.

In a further embodiment, the one or more additional sweeteners is selected from the group consisting of mogroside IA, mogroside IE, 11-oxomogroside IA, mogroside II, mogroside II A, mogroside II B, mogroside II E, 7-oxomogroside II E, mogroside III, Mogroside IIIe, 11-deoxymogroside III, mogroside IV, 11-oxomogroside IV, 11-oxomogroside IV A, 11-deoxymogroside V, 7-oxomogroside V, 11-oxomogroside V, isomogroside V, mogroside VI, mogrol, 11-oxomogrol, the 1,6-α isomer of siamenoside I, monk fruit extract, and combinations thereof.

In a particular embodiment, the one or more additional sweeteners is rebaudioside M (13-[2-O-β-D-glucopyranosyl-3-O-β-D-glucopyranosyl-β-D-glucopyranosyl)oxy] Ent Kaur-16-end-19-oil acid-[2-O-β-D-glucopyranosyl-3-O-β-D-glycopyranosyl) ester having the formula:

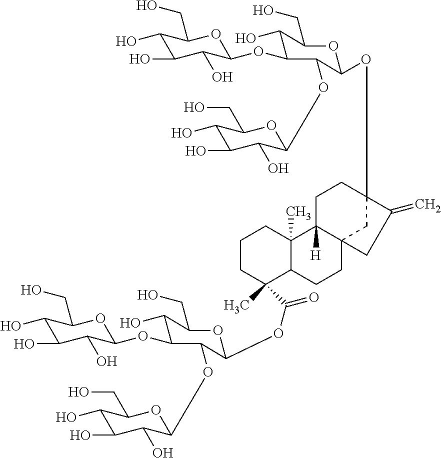

Reb M may be provided in a purified or unpurified form, i.e., as part of a naturally occurring mixture that contains Reb M. In one embodiment, Reb M can be obtained from a Stevia extract by any suitable purification method. Suitable purification methods are known in the art, including, but not limited to, column chromatography, recrystallization, phase separation, extraction, high performance liquid chromatography and combinations thereof.

In another embodiment, the additional sweetener is a steviol glycoside composition. An exemplary steviol glycoside composition is A95, which contains primarily reb D and reb M with minor amounts of one or more of the following: Reb E, Reb O, Reb N, Reb A, Stevioside, Reb C and Reb B. Methods of obtaining A95 are provided in WO 2017/059414, incorporated herein by reference. An exemplary A95 blend is provided in Example 7 herein.

The amount of Reb M in the sweeter composition or taste modifying composition may vary. In one embodiment, Reb M is present in a sweetener composition in any amount to impart the desired sweetness when the sweetener composition is added to a consumable e.g., a beverage). In a particular embodiment, the desired sweetness of the consumable is greater than about 10 degrees Brix.

In one embodiment, the sweetener composition contains Reb M in an amount effective to provide sweetness equivalent from about 1 to 12 degrees Brix when added to a consumable (e.g., a beverage), such as, for example, from about 2 to about 9 degrees Brix, from about 3 to about 8 degrees Brix, from about 4 to about 7 degrees Brix, or about 5 degrees Brix.

In a particular embodiment, Reb M is present in an effective amount to provide a sucrose equivalence (SE) of about 8 or less, such as for example, about 7, about 6.5, about 6, about 5.5, or about 5 SE.

In another particular embodiment, Reb M is present in an effective amount to provide a sucrose equivalence of about 8 or greater, such as, for example, about 9, about 9.5, about 10, about 10.5, about 11, about 11.5, about 12, about 12.5, about 13, about 13.5, about 14, about 14.5 or about 15.

In one embodiment, Reb M is present in the flavor modifying composition in any amount to impart the desired flavor when the flavor modifying composition is added to a flavor modifiable composition (e.g., a beverage). In a particular embodiment, the desired flavor is a more sugar-like temporal or taste profile.

In one embodiment, the Reb M and the brazzein produce a synergistic effect, e.g., synergistic sweetness, i.e., the sweetness of combination is greater than the sum of the individual sweeteners. In a particular embodiment, the Reb M and the brazzein product an effect that would be unexpected by one of skill in the art.

Reb M may be provided in a purified form or as a component of a mixture containing Reb M and one or more additional components. In one embodiment, Reb X is provided as a component of a mixture. In a particular embodiment, the mixture is a Stevia extract. The Stevia extract may contain Reb M in an amount that ranges from about 5% to about 100% by weight on a dry basis, such as, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100%. In still further embodiments, the Stevia extract contains Reb M in an amount greater than about 90% by weight on a dry basis, for example, greater than about 91%, greater than about 92%, greater than about 93%, greater than about 94%, greater than about 95%, greater than about 96%, greater than about 97%, greater than about 98% and greater than about 99%.

In one embodiment, Reb M is provided as a component of a steviol glycoside mixture, i.e., a mixture of steviol glycosides wherein the remainder of the non-Reb M portion of the mixture is comprised entirely of steviol glycosides. The identities of steviol glycosides are known the art and include, but are not limited to, steviol, steviol monoside, rubososide, steviolbiocide, stevioside, rebaudioside A, rebaudioside B, rebaudioside C, rebaudioside D, rebaudioside E, rebaudioside F and dulcoside A. The steviol glycoside mixture may contain from about 5% to about 100% Reb M by weight on a dry basis. For example, a steviol glycoside mixture may contain from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100% Reb M by weight on a dry basis. In still further embodiments, the steviol glycoside mixture may contain greater than about 90%, for example, greater than about 91%, greater than about 92%, greater than about 93%, greater than about 94%, greater than about 95%, greater than about 96%, greater than about 97%, greater than about 98% and greater than about 99% Reb M by weight on a dry basis.

RebM80 refers to a Stevia extract or steviol glycoside composition having about 80% Reb M by weight.

In a particular embodiment, the one or more additional sweeteners is rebaudioside A.

Reb A may be provided in a purified or unpurified form, i.e., as part of a naturally occurring mixture that contains Reb a. In one embodiment, Reb A can be obtained from a Stevia extract by any suitable purification method Suitable purification methods are known in the art, including, but not limited to, column chromatography, recrystallization, phase separation, extraction, high performance liquid chromatography and combinations thereof.

Reb A may be provided in a purified form or as a component of a mixture containing Reb A and one or more additional components. In one embodiment, Reb A is provided as a component of a mixture. In a particular embodiment, the mixture is a Stevia extract. The Stevia extract may contain Reb A in an amount that ranges from about 5% to about 100% by weight on a dry basis, such as, for example, from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100%. In still further embodiments, the Stevia extract contains Reb A in an amount greater than about 90% by weight on a dry basis, for example, greater than about 91%, greater than about 92%, greater than about 93%, greater than about 94%, greater than about 95%, greater than about 96%, greater than about 97%, greater than about 98% and greater than about 99%.

In one embodiment, Reb A is provided as a component of a steviol glycoside mixture, i.e., a mixture of steviol glycosides wherein the remainder of the non-Reb A portion of the mixture is comprised entirely of steviol glycosides. The steviol glycoside mixture may contain from about 5% to about 100% Reb A weight on a dry basis. For example, a steviol glycoside mixture may contain from about 10% to about 100%, from about 20% to about 100%, from about 30% to about 100%, from about 40% to about 100%, from about 50% to about 100%, from about 60% to about 100%, from about 70% to about 100%, from about 80% to about 100% and from about 90% to about 100% Reb A by weight on a dry basis. In still further embodiments, the steviol glycoside mixture may contain greater than about 90%, for example, greater than about 91%, greater than about 92%, greater than about 93%, greater than about 94%, greater than about 95%, greater than about 96%, greater than about 97%, greater than about 98% and greater than about 99% Reb A by weight on a dry basis.

The amount of Reb A in the sweetener composition or taste modifying composition may vary. In one embodiment, Reb A is present in the sweetener composition in any amount to impart the desired sweetness when the sweetener composition is added to a sweetenable composition. In a particular embodiment, the desired sweetness of the sweetened composition is greater than about 10 degrees Brix.

In one embodiment, the Reb A and the brazzein produce a synergistic effect, e.g., synergistic sweetness, i.e., the sweetness of combination is greater than the sum of the individual sweeteners. In certain embodiments, the Reb A and brazzein produce an effect that one of skill in the art would not have expected.

In a particular embodiment, Reb A is present in an effective amount to provide a sucrose equivalence (SE) of about 8 or less, such as for example, about 7, about 6.5, about 6, about 5.5, or about 5 SE.

In another particular embodiment, Reb A is present in an effective amount to provide a sucrose equivalence (SE) of about 8 or greater, such as, for example, about 9, about 9.5, about 10, about 10.5, about 11, about 11.5, about 12, about 12.5, about 13, about 13.5, about 14, about 14.5 or about 15.

In another particular embodiment, Reb A is present in an effective amount to provide a sucrose equivalence of greater than about 8 SE, e.g., about 9 SE, about 9.5 SE, about 10 SE.

In one embodiment, Reb A is present in the flavor modifying composition in any amount to impart the desired taste when the taste modifying composition is added to a taste modifiable composition (e.g., a beverage). In a particular embodiment, the desired taste is a sugar-like taste.

In a particular embodiment, the brazzein and Reb A produce a synergistic effect. In one embodiment, the brazzein and Reb A produce an effect that would been unexpected to one of skill in the art.

In another embodiment, the one or more additional sweetener may be a carbohydrate sweetener. Non-limiting examples of suitable carbohydrate sweeteners include sucrose, fructose, glucose, erythritol, maltitol, lactitol, sorbitol, mannitol, xylitol, D-tagatose, trehalose, galactose, rhamnose, cyclodextrin (e.g., α-cyclodextrin, β-cyclodextrin, and γ-cyclodextrin), ribulose, threose, arabinose, xylose, lyxose, allose, altrose, mannose, idose, lactose, maltose, invert sugar, isotrehalose, neotrehalose, palatinose or isomaltulose, erythrose, deoxyribose, gulose, idose, talose, erythrulose, xylulose, psicose, turanose, cellobiose, glucosamine, mannosamine, fucose, fuculose, glucuronic acid, gluconic acid, glucono-lactone, abequose, galactosamine, xylo-oligosaccharides (xylotriose, xylobiose and the like), gentio-oligosaccharides (gentiobiose, gentiotriose, gentiotetraose and the like), galacto-oligosaccharides, sorbose, ketotriose (dehydroxyacetone), aldotriose (glyceraldehyde), nigero-oligosaccharides, fructooligosaccharides (kestose, nystose and the like), maltotetraose, maltotriol, tetrasaccharides, mannan-oligosaccharides, malto-oligosaccharides (maltotriose, maltotetraose, maltopentaose, maltohexaose, maltoheptaose and the like), dextrins, lactulose, melibiose, raffinose, rhamnose, ribose, isomerized liquid sugars such as high fructose corn/starch syrup (HFCS/HFSS) (e.g., HFCS55, HFCS42, or HFCS90), coupling sugars, soybean oligosaccharides, glucose syrup and combinations thereof.

In other embodiments, the at least one additional sweetener is a synthetic sweetener. As used herein, the phrase “synthetic sweetener” refers to any composition which is not found naturally in nature and characteristically has a sweetness potency greater than sucrose, fructose, or glucose, yet has less calories. Non-limiting examples of synthetic high-potency sweeteners suitable for embodiments of this disclosure include sucralose, potassium acesulfame, aspartame, alitame, saccharin, neohesperidin dihydrochalcone, cyclamate, neotame, advantame, glucosylated steviol glycosides (GSGs) and combinations thereof.

The sweetener compositions can be customized to obtain a desired calorie content. For example, sweetener compositions can be “high-calorie”, such that they impart the desired sweetness when added to a sweetenable composition (such as, for example, as beverage) and have about 120 calories per 8 oz serving.

The sweetener compositions can be customized to obtain a desired calorie content. For example, sweetener compositions can be “mid-calorie”, such that they impart the desired sweetness when added to a sweetenable composition (such as, for example, as beverage) and have about 80 calories per 8 oz serving.

For example, sweetener compositions can be “low-calorie”, such that they impart the desired sweetness when added to a sweetenable composition (such as, for example, as beverage) and have less than 40 calories per 8 oz serving.

In other embodiments, the sweetener compositions can be “zero-calorie”, such that they impart the desired sweetness when added to a sweetenable composition (such as, for example, a beverage) and have less than 5 calories per 8 oz. serving.

Additives

In addition to brazzein (or analog thereof) and, optionally, one or more additional sweeteners (e.g., one or more steviol glycosides), the sweetener compositions or flavor modifying compositions disclosed herein can optionally include additional additives, detailed herein below. In some embodiments, the sweetener composition contains additives including, but not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof. In some embodiments, the additives act to improve the temporal and flavor profile of the sweetener to provide a sweetener composition with a taste similar to sucrose.

In one embodiment, the sweetener compositions or flavor modifying compositions contain one or more polyols. The term “polyol”, as used herein, refers to a molecule that contains more than one hydroxyl group. A polyol may be a diol, triol, or a tetraol which contains 2, 3, and 4 hydroxyl groups respectively. A polyol also may contain more than 4 hydroxyl groups, such as a pentaol, hexaol, heptaol, or the like, which contain 5, 6, or 7 hydroxyl groups, respectively. Additionally, a polyol also may be a sugar alcohol, polyhydric alcohol, or polyalcohol which is a reduced form of carbohydrate, wherein the carbonyl group (aldehyde or ketone, reducing sugar) has been reduced to a primary or secondary hydroxyl group.

Non-limiting examples of polyols in some embodiments include erythritol, maltitol, mannitol, sorbitol, lactitol, xylitol, isomalt, propylene glycol, glycerol (glycerin), threitol, galactitol, palatinose, reduced isomalto-oligosaccharides, reduced xylo-oligosaccharides, reduced gentio-oligosaccharides, reduced maltose syrup, reduced glucose syrup, and sugar alcohols or any other carbohydrates capable of being reduced which do not adversely affect taste.

Suitable sweet taste improving amino acid additives include, but are not limited to, aspartic acid, arginine, glycine, glutamic acid, proline, threonine, theanine, cysteine, cystine, alanine, valine, tyrosine, leucine, arabinose, trans-4-hydroxyproline, isoleucine, asparagine, serine, lysine, histidine, ornithine, methionine, carnitine, aminobutyric acid (α-, β-, and/or δ-isomers), glutamine, hydroxyproline, taurine, norvaline, sarcosine, and their salt forms such as sodium or potassium salts or acid salts. The sweet taste improving amino acid additives also may be in the D- or L-configuration and in the mono-, di-, or tri-form of the same or different amino acids. Additionally, the amino acids may be α-, β-, γ- and/or δ-isomers if appropriate. Combinations of the foregoing amino acids and their corresponding salts (e.g., sodium, potassium, calcium, magnesium salts or other alkali or alkaline earth metal salts thereof, or acid salts) also are suitable sweet taste improving additives in some embodiments. The amino acids may be natural or synthetic. The amino acids also may be modified. Modified amino acids refers to any amino acid wherein at least one atom has been added, removed, substituted, or combinations thereof (e.g., N-alkyl amino acid, N-acyl amino acid, or N-methyl amino acid). Non-limiting examples of modified amino acids include amino acid derivatives such as trimethyl glycine, N-methyl-glycine, and N-methyl-alanine. As used herein, modified amino acids encompass both modified and unmodified amino acids. As used herein, amino acids also encompass both peptides and polypeptides (e.g., dipeptides, tripeptides, tetrapeptides, and pentapeptides) such as glutathione and L-alanyl-L-glutamine. Suitable sweet taste improving polyamino acid additives include poly-L-aspartic acid, poly-L-lysine (e.g., poly-L-α-lysine or poly-L-ε-lysine), poly-L-ornithine (e.g., poly-L-□α-ornithine or poly-L-□ε-ornithine), poly-L-arginine, other polymeric forms of amino acids, and salt forms thereof (e.g., calcium, potassium, sodium, or magnesium salts such as L-glutamic acid mono sodium salt). The sweet taste improving poly-amino acid additives also may be in the D- or L-configuration. Additionally, the poly-amino acids may be α-, β-, γ-, δ-, and ε-isomers if appropriate. Combinations of the foregoing poly-amino acids and their corresponding salts (e.g., sodium, potassium, calcium, magnesium salts or other alkali or alkaline earth metal salts thereof or acid salts) also are suitable sweet taste improving additives in some embodiments. The poly-amino acids described herein also may comprise co-polymers of different amino acids. The poly-amino acids may be natural or synthetic. The poly-amino acids also may be modified, such that at least one atom has been added, removed, substituted, or combinations thereof (e.g., N-alkyl poly-amino acid or N-acyl poly-amino acid). As used herein, poly-amino acids encompass both modified and unmodified poly-amino acids. For example, modified poly-amino acids include, but are not limited to, poly-amino acids of various molecular weights (MW), such as poly-L-α-lysine with a MW of 1,500, MW of 6,000, MW of 25,200, MW of 63,000, MW of 83,000, or MW of 300,000.

Suitable sugar acid additives include, but are not limited to, aldonic, uronic, aldaric, alginic, gluconic, glucuronic, glucaric, galactaric, galacturonic, and salts thereof (e.g., sodium, potassium, calcium, magnesium salts or other physiologically acceptable salts), and combinations thereof.

Suitable nucleotide additives include, but are not limited to, inosine monophosphate (“IMP”), guanosine monophosphate (“GMP”), adenosine monophosphate (“AMP”), cytosine monophosphate (CMP), uracil monophosphate (UMP), inosine diphosphate, guanosine diphosphate, adenosine diphosphate, cytosine diphosphate, uracil diphosphate, inosine triphosphate, guanosine triphosphate, adenosine triphosphate, cytosine triphosphate, uracil triphosphate, alkali or alkaline earth metal salts thereof, and combinations thereof. The nucleotides described herein also may comprise nucleotide-related additives, such as nucleosides or nucleic acid bases (e.g., guanine, cytosine, adenine, thymine, uracil). In particular embodiments, the nucleotide is present in the sweetener composition in an amount from about 5 ppm to about 1,000 ppm.

Suitable organic acid additives include any compound which comprises a —COOH moiety, such as, for example, C2-C30 carboxylic acids, substituted hydroxyl C2-C30 carboxylic acids, benzoic acid, substituted benzoic acids (e.g., 2,4-dihydroxybenzoic acid), substituted cinnamic acids, hydroxyacids, substituted hydroxybenzoic acids, substituted cyclohexyl carboxylic acids, tannic acid, lactic acid, tartaric acid, citric acid, gluconic acid, glucoheptonic acids, adipic acid, hydroxycitric acid, malic acid, fruitaric acid (a blend of malic, fumaric, and tartaric acids), fumaric acid, maleic acid, succinic acid, chlorogenic acid, salicylic acid, creatine, caffeic acid, bile acids, acetic acid, ascorbic acid, alginic acid, erythorbic acid, polyglutamic acid, glucono delta lactone, and their alkali or alkaline earth metal salt derivatives thereof. In addition, the organic acid additives also may be in either the D- or L-configuration.

Suitable organic acid additive salts include, but are not limited to, sodium, calcium, potassium, and magnesium salts of all organic acids, such as salts of citric acid, malic acid, tartaric acid, fumaric acid, lactic acid (e.g., sodium lactate), alginic acid (e.g., sodium alginate), ascorbic acid (e.g., sodium ascorbate), benzoic acid (e.g., sodium benzoate or potassium benzoate), and adipic acid. The examples of the sweet taste improving organic acid additives described optionally may be substituted with at least one group chosen from hydrogen, alkyl, alkenyl, alkynyl, halo, haloalkyl, carboxyl, acyl, acyloxy, amino, amido, carboxyl derivatives, alkylamino, dialkylamino, arylamino, alkoxy, aryloxy, nitro, cyano, sulfo, thiol, imine, sulfonyl, sulfenyl, sulfinyl, sulfamyl, carboxalkoxy, carboxamido, phosphonyl, phosphinyl, phosphoryl, phosphino, thioester, thioether, anhydride, oximino, hydrazino, carbamyl, phosphor or phosphonato. In particular embodiments, the organic acid additive is present in the sweetener composition in an amount from about 10 ppm to about 5,000 ppm.

Suitable inorganic acid additives include, but are not limited to, phosphoric acid, phosphorous acid, polyphosphoric acid, hydrochloric acid, sulfuric acid, carbonic acid, sodium dihydrogen phosphate, and alkali or alkaline earth metal salts thereof (e.g., inositol hexaphosphate Mg/Ca).

Suitable bitter compound additives include, but are not limited to, caffeine, quinine, urea, bitter orange oil, naringin, quassia, and salts thereof.

Suitable flavorant and flavoring ingredient additives for include, but are not limited to, vanillin, vanilla extract, mango extract, cinnamon, Citrus, coconut, ginger, viridiflorol, almond, menthol (including menthol without mint), grape skin extract, and grape seed extract. “Flavorant” and “flavoring ingredient” are synonymous and can include natural or synthetic substances or combinations thereof. Flavorants also include any other substance which imparts flavor and may include natural or non-natural (synthetic) substances which are safe for human or animals when used in a generally accepted range. Non-limiting examples of proprietary flavorants include Döhler™ Natural Flavoring Sweetness Enhancer K14323 (Döhler™, Darmstadt, Germany), Symrise™ Natural Flavor Mask for Sweeteners 161453 and 164126 (Symrise™, Holzminden, Germany), Natural Advantage™ Bitterness Blockers 1, 2, 9 and 10 (Natural Advantage™, Freehold, N.J., U.S.A.), and Sucramask™ (Creative Research Management, Stockton, Calif., U.S.A.).

Suitable polymer additives include, but are not limited to, chitosan, pectin, pectic, pectinic, polyuronic, polygalacturonic acid, starch, food hydrocolloid or crude extracts thereof (e.g., gum acacia senegal (Fibergum™), gum acacia seyal, carageenan), poly-L-lysine (e.g., poly-L-α-lysine or poly-L-ε-lysine), poly-L-ornithine (e.g., poly-L-α-ornithine or poly-L-ε-ornithine), polypropylene glycol, polyethylene glycol, poly(ethylene glycol methyl ether), polyarginine, polyaspartic acid, polyglutamic acid, polyethylene imine, alginic acid, sodium alginate, propylene glycol alginate, and sodium polyethyleneglycolalginate, sodium hexametaphosphate and its salts, and other cationic polymers and anionic polymers.

Suitable protein or protein hydrolysate additives include, but are not limited to, bovine serum albumin (BSA), whey protein (including fractions or concentrates thereof such as 90% instant whey protein isolate, 34% whey protein, 50% hydrolyzed whey protein, and 80% whey protein concentrate), soluble rice protein, soy protein, protein isolates, protein hydrolysates, reaction products of protein hydrolysates, glycoproteins, and/or proteoglycans containing amino acids (e.g., glycine, alanine, serine, threonine, asparagine, glutamine, arginine, valine, isoleucine, leucine, norvaline, methionine, proline, tyrosine, hydroxyproline, and the like), collagen (e.g., gelatin), partially hydrolyzed collagen (e.g., hydrolyzed fish collagen), and collagen hydrolysates (e.g., porcine collagen hydrolysate).

Suitable surfactant additives include, but are not limited to, polysorbates (e.g., polyoxyethylene sorbitan monooleate (polysorbate 80), polysorbate 20, polysorbate 60), sodium dodecylbenzenesulfonate, dioctyl sulfosuccinate or dioctyl sulfosuccinate sodium, sodium dodecyl sulfate, cetylpyridinium chloride (hexadecylpyridinium chloride), hexadecyltrimethylammonium bromide, sodium cholate, carbamoyl, choline chloride, sodium glycocholate, sodium taurodeoxycholate, lauric arginate, sodium stearoyl lactylate, sodium taurocholate, lecithins, sucrose oleate esters, sucrose stearate esters, sucrose palmitate esters, sucrose laurate esters, and other emulsifiers, and the like.

Suitable flavonoid additives are classified as flavonols, flavones, flavanones, flavan-3-ols, isoflavones, or anthocyanidins. Non-limiting examples of flavonoid additives include, but are not limited to, catechins (e.g., green tea extracts such as Polyphenon™ 60, Polyphenon™ 30, and Polyphenon™ 25 (Mitsui Norin Co., Ltd., Japan), polyphenols, rutins (e.g., enzyme modified rutin Sanmelin™ AO (San-fi Gen F.F.I., Inc., Osaka, Japan)), neohesperidin, naringin, neohesperidin dihydrochalcone, and the like.

Suitable alcohol additives include, but are not limited to, ethanol.

Suitable astringent compound additives include, but are not limited to, tannic acid, europium chloride (EuCl₃), gadolinium chloride (GdCl₃), terbium chloride (TbCl₃), alum, tannic acid, and polyphenols (e.g., tea polyphenols). In particular embodiments, the astringent additive is present in an amount from about 10 ppm to about 5,000 ppm.

In particular embodiments, sweetener compositions or flavor modifying compositions comprise brazzein (or analog thereof), optionally in combination with one or more steviol glycosides (e.g., Reb M, Reb A), a polyol selected from erythritol, maltitol, mannitol, xylitol, sorbitol, and combinations thereof; and optionally at least one additional sweetener and/or functional ingredient. In a particular embodiment, the polyol is erythritol. The steviol glycoside (e.g., Reb M, Reb A) can be provided as a pure compound or as part of a Stevia extract or steviol glycoside mixture, as described above. The steviol glycoside (e.g., Reb M, Reb A) can be present in an amount from about 5% to about 100% by weight on a dry basis in either a steviol glycoside mixture or a Stevia extract.

In particular embodiments, the sweetener compositions or flavor modifying compositions comprise brazzein (or analog thereof), optionally in combination with one or more steviol glycosides (e.g., Reb M, Reb A); a carbohydrate sweetener selected from sucrose, fructose, glucose, maltose and combinations thereof; and optionally at least one additional sweetener and/or functional ingredient. The steviol glycoside (e.g., Reb M, Reb A) can be provided as a pure compound or as part of a Stevia extract or steviol glycoside mixture, as described above. The steviol glycoside (e.g., Reb M, Reb A) can be present in an amount from about 5% to about 100% by weight on a dry basis in either a steviol glycoside mixture or a Stevia extract.

In particular embodiments, the sweetener compositions or flavor modifying compositions comprise brazzein (or analog thereof), optionally in combination with one or more steviol glycosides (e.g., Reb M, Reb A); an amino acid selected from glycine, alanine, proline and combinations thereof; and optionally at least one additional sweetener and/or functional ingredient. The steviol glycoside can be provided as a pure compound or as part of a Stevia extract or steviol glycoside mixture, as described above. The steviol glycoside (e.g., Reb M, Reb A) can be present in an amount from about 5% to about 100% by weight on a dry basis in either a steviol glycoside mixture or a Stevia extract.

In particular embodiments, sweetener compositions or flavor modifying compositions comprise brazzein (or analog thereof), optionally in combination with one or more steviol glycosides (e.g., Reb M, Reb A), a salt selected from sodium chloride, magnesium chloride, potassium chloride, calcium chloride and combinations thereof; and optionally at least one additional sweetener and/or functional ingredient. The steviol glycoside (e.g., Reb M, Reb A) can be provided as a pure compound or as part of a Stevia extract or steviol glycoside mixture, as described above.

Functional Ingredients

The sweetener compositions or flavor modifying compositions disclosed herein can also contain one or more functional ingredients, which provide a real or perceived heath benefit to the composition. Functional ingredients include, but are not limited to, saponins, antioxidants, dietary fiber sources, fatty acids, vitamins, glucosamine, minerals, preservatives, hydration agents, probiotics, prebiotics, weight management agents, osteoporosis management agents, phytoestrogens, long chain primary aliphatic saturated alcohols, phytosterols and combinations thereof.

Examples of suitable antioxidants for embodiments of this invention include, but are not limited to, vitamins, vitamin cofactors, minerals, hormones, carotenoids, carotenoid terpenoids, non-carotenoid terpenoids, flavonoids, flavonoid polyphenolics (e.g., bioflavonoids), flavonols, flavones, phenols, polyphenols, esters of phenols, esters of polyphenols, nonflavonoid phenolics, isothiocyanates, and combinations thereof. In some embodiments, the antioxidant is vitamin A, vitamin C, vitamin E, ubiquinone, mineral selenium, manganese, melatonin, α-carotene, β-carotene, lycopene, lutein, zeanthin, crypoxanthin, reservatol, eugenol, quercetin, catechin, gossypol, hesperetin, curcumin, ferulic acid, thymol, hydroxytyrosol, tumeric, thyme, olive oil, lipoic acid, glutathinone, gutamine, oxalic acid, tocopherol-derived compounds, butylated hydroxyanisole (BHA), butylated hydroxytoluene (BHT), ethylenediaminetetraacetic acid (EDTA), tert-butylhydroquinone, acetic acid, pectin, tocotrienol, tocopherol, coenzyme Q10, zeaxanthin, astaxanthin, canthaxantin, saponins, limonoids, kaempfedrol, myricetin, isorhamnetin, proanthocyanidins, quercetin, rutin, luteolin, apigenin, tangeritin, hesperetin, naringenin, erodictyol, flavan-3-ols (e.g., anthocyanidins), gallocatechins, epicatechin and its gallate forms, epigallocatechin and its gallate forms (ECGC) theaflavin and its gallate forms, thearubigins, isoflavone phytoestrogens, genistein, daidzein, glycitein, anythocyanins, cyaniding, delphinidin, malvidin, pelargonidin, peonidin, petunidin, ellagic acid, gallic acid, salicylic acid, rosmarinic acid, cinnamic acid and its derivatives (e.g., ferulic acid), chlorogenic acid, chicoric acid, gallotannins, ellagitannins, anthoxanthins, betacyanins and other plant pigments, silymarin, citric acid, lignan, antinutrients, bilirubin, uric acid, R-α-lipoic acid, N-acetylcysteine, emblicanin, and phytic acid, or combinations thereof. In alternate embodiments, the antioxidant is a synthetic antioxidant such as butylated hydroxytolune or butylated hydroxyanisole, for example. Other sources of suitable antioxidants for embodiments of this invention include, but are not limited to, fruits, vegetables, tea, cocoa, chocolate, spices, herbs, rice, organ meats from livestock, yeast, whole grains, or cereal grains.

Particular antioxidants belong to the class of phytonutrients called polyphenols (also known as “polyphenolics”), which are a group of chemical substances found in plants, characterized by the presence of more than one phenol group per molecule. Suitable polyphenols for embodiments of this invention, include catechins, proanthocyanidins, procyanidins, anthocyanins, quercerin, rutin, reservatrol, isoflavones, curcumin, punicalagin, ellagitannin, hesperidin, naringin, Citrus flavonoids, chlorogenic acid, other similar materials, and combinations thereof.

In particular embodiments, the antioxidant is a catechin such as, for example, epigallocatechin gallate (EGCG). Suitable sources of catechins for embodiments of this invention include, but are not limited to, green tea, white tea, black tea, oolong tea, chocolate, cocoa, red wine, grape seed, red grape skin, purple grape skin, red grape juice, purple grape juice, berries, pycnogenol, and red apple peel.

In some embodiments, the antioxidant is chosen from proanthocyanidins, procyanidins or combinations thereof. Suitable sources of proanthocyanidins and procyanidins for embodiments of this invention include, but are not limited to, red grapes, purple grapes, cocoa, chocolate, grape seeds, red wine, cacao beans, cranberry, apple peel, plum, blueberry, black currants, choke berry, green tea, sorghum, cinnamon, barley, red kidney bean, pinto bean, hops, almonds, hazelnuts, pecans, pistachio, pycnogenol, and colorful berries.

In particular embodiments, the antioxidant is an anthocyanin. Suitable sources of anthocyanins for embodiments of this invention include, but are not limited to, red berries, blueberries, bilberry, cranberry, raspberry, cherry, pomegranate, strawberry, elderberry, choke berry, red grape skin, purple grape skin, grape seed, red wine, black currant, red currant, cocoa, plum, apple peel, peach, red pear, red cabbage, red onion, red orange, and blackberries.

In some embodiments, the antioxidant is chosen from quercetin, rutin or combinations thereof. Suitable sources of quercetin and rutin for embodiments of this invention include, but are not limited to, red apples, onions, kale, bog whortleberry, lingonberrys, chokeberry, cranberry, blackberry, blueberry, strawberry, raspberry, black currant, green tea, black tea, plum, apricot, parsley, leek, broccoli, chili pepper, berry wine, and ginkgo.

In some embodiments, the antioxidant is resveratrol. Suitable sources of resveratrol for embodiments of this invention include, but are not limited to, red grapes, peanuts, cranberry, blueberry, bilberry, mulberry, Japanese Itadori tea, and red wine.

In particular embodiments, the antioxidant is an isoflavone. Suitable sources of isoflavones for embodiments of this invention include, but are not limited to, soy beans, soy products, legumes, alfalfa sprouts, chickpeas, peanuts, and red clover.

In some embodiments, the antioxidant is curcumin. Suitable sources of curcumin for embodiments of this invention include, but are not limited to, turmeric and mustard.

In particular embodiments, the antioxidant is chosen from punicalagin, ellagitannin or combinations thereof. Suitable sources of punicalagin and ellagitannin for embodiments of this invention include, but are not limited to, pomegranate, raspberry, strawberry, walnut, and oak-aged red wine.

In some embodiments, the antioxidant is a Citrus flavonoid, such as hesperidin or naringin. Suitable sources of Citrus flavonoids, such as hesperidin or naringin, for embodiments of this invention include, but are not limited to, oranges, grapefruits, and Citrus juices.

In particular embodiments, the antioxidant is chlorogenic acid. Suitable sources of chlorogenic acid for embodiments of this invention include, but are not limited to, green coffee, yerba mate, red wine, grape seed, red grape skin, purple grape skin, red grape juice, purple grape juice, apple juice, cranberry, pomegranate, blueberry, strawberry, sunflower, Echinacea, pycnogenol, and apple peel.

Suitable dietary fibers include, but are not limited to, non-starch polysaccharides, lignin, cellulose, methylcellulose, the hemicelluloses, β-glucans, pectins, gums, mucilage, waxes, inulins, oligosaccharides, fructooligosaccharides, cyclodextrins, chitins, and combinations thereof.

Food sources of dietary fiber include, but are not limited to, grains, legumes, fruits, and vegetables. Grains providing dietary fiber include, but are not limited to, oats, rye, barley, and wheat. Legumes providing fiber include, but are not limited to, peas and beans such as soybeans. Fruits and vegetables providing a source of fiber include, but are not limited to, apples, oranges, pears, bananas, berries, tomatoes, green beans, broccoli, cauliflower, carrots, potatoes, celery. Plant foods such as bran, nuts, and seeds (such as flax seeds) are also sources of dietary fiber. Parts of plants providing dietary fiber include, but are not limited to, the stems, roots, leaves, seeds, pulp, and skin.

Fatty acids any straight chain monocarboxylic acid and includes saturated fatty acids, unsaturated fatty acids, long chain fatty acids, medium chain fatty acids, short chain fatty acids, fatty acid precursors (including omega-9 fatty acid precursors), and esterified fatty acids. As used herein, “long chain polyunsaturated fatty acid” refers to any polyunsaturated carboxylic acid or organic acid with a long aliphatic tail. Suitable omega-3 fatty acids include, but are not limited to, linolenic acid, alpha-linolenic acid, eicosapentaenoic acid, docosahexaenoic acid, stearidonic acid, eicosatetraenoic acid and combinations thereof.

Suitable omega-6 fatty acids include, but are not limited to, linoleic acid, gamma-linolenic acid, dihommo-gamma-linolenic acid, arachidonic acid, eicosadienoic acid, docosadienoic acid, adrenic acid, docosapentaenoic acid and combinations thereof. Suitable esterified fatty acids for embodiments of the present invention include, but are not limited to, monoacylgycerols containing omega-3 and/or omega-6 fatty acids, diacylgycerols containing omega-3 and/or omega-6 fatty acids, or triacylgycerols containing omega-3 and/or omega-6 fatty acids and combinations thereof.

Suitable vitamins include, vitamin A, vitamin D, vitamin E, vitamin K, vitamin B1, vitamin B2, vitamin B3, vitamin B5, vitamin B6, vitamin B7, vitamin B9, vitamin B12, and vitamin C. Various other compounds have been classified as vitamins by some authorities. These compounds may be termed pseudo-vitamins and include, but are not limited to, compounds such as ubiquinone (coenzyme Q10), pangamic acid, dimethylglycine, taestrile, amygdaline, flavanoids, para-aminobenzoic acid, adenine, adenylic acid, and s-methylmethionine. As used herein, the term vitamin includes pseudo-vitamins.

Minerals are selected from bulk minerals, trace minerals or combinations thereof. Non-limiting examples of bulk minerals include calcium, chlorine, magnesium, phosphorous, potassium, sodium, and sulfur. Non-limiting examples of trace minerals include chromium, cobalt, copper, fluorine, iron, manganese, molybdenum, selenium, zinc, and iodine. Although iodine generally is classified as a trace mineral, it is required in larger quantities than other trace minerals and often is categorized as a bulk mineral.

In other particular embodiments of this invention, the mineral is a trace mineral, believed to be necessary for human nutrition, non-limiting examples of which include bismuth, boron, lithium, nickel, rubidium, silicon, strontium, tellurium, tin, titanium, tungsten, and vanadium.

Preservatives are selected from antimicrobials, antioxidants, antienzymatics or combinations thereof. Non-limiting examples of antimicrobials include sulfites, propionates, benzoates, sorbates, nitrates, nitrites, bacteriocins, salts, sugars, acetic acid, dimethyl dicarbonate (DMDC), ethanol, and ozone. Sulfites include, but are not limited to, sulfur dioxide, sodium bisulfite, and potassium hydrogen sulfite. Propionates include, but are not limited to, propionic acid, calcium propionate, and sodium propionate. Benzoates include, but are not limited to, sodium benzoate and benzoic acid. Sorbates include, but are not limited to, potassium sorbate, sodium sorbate, calcium sorbate, and sorbic acid. Nitrates and nitrites include, but are not limited to, sodium nitrate and sodium nitrite. In yet another particular embodiment, the at least one preservative is a bacteriocin, such as, for example, nisin. In another particular embodiment, the preservative is ethanol. In still another particular embodiment, the preservative is ozone. Antienzymatics suitable for use as preservatives in particular embodiments of the invention include ascorbic acid, citric acid, and metal chelating agents such as ethylenediaminetetraacetic acid (EDTA).

Hydration products can be electrolytes, non-limiting examples of which include sodium, potassium, calcium, magnesium, chloride, phosphate, bicarbonate, and combinations thereof. Suitable electrolytes for use in particular embodiments of this invention are also described in U.S. Pat. No. 5,681,569, the disclosure of which is expressly incorporated herein by reference. Non-limiting examples of salts for use in particular embodiments include chlorides, carbonates, sulfates, acetates, bicarbonates, citrates, phosphates, hydrogen phosphates, tartrates, sorbates, citrates, benzoates, or combinations thereof.

In particular embodiments of this invention, the hydration product is a carbohydrate to supplement energy stores burned by muscles. Suitable carbohydrates for use in particular embodiments of this invention are described in U.S. Pat. Nos. 4,312,856, 4,853,237, 5,681,569, and 6,989,171, the disclosures of which are expressly incorporated herein by reference. Non-limiting examples of suitable carbohydrates include monosaccharides, disaccharides, oligosaccharides, complex polysaccharides or combinations thereof. Non-limiting examples of suitable types of monosaccharides for use in particular embodiments include trioses, tetroses, pentoses, hexoses, heptoses, octoses, and nonoses. Non-limiting examples of specific types of suitable monosaccharides include glyceraldehyde, dihydroxyacetone, erythrose, threose, erythrulose, arabinose, lyxose, ribose, xylose, ribulose, xylulose, allose, altrose, galactose, glucose, gulose, idose, mannose, talose, fructose, psicose, sorbose, tagatose, mannoheptulose, sedoheltulose, octolose, and sialose. Non-limiting examples of suitable disaccharides include sucrose, lactose, and maltose. Non-limiting examples of suitable oligosaccharides include saccharose, maltotriose, and maltodextrin. In other particular embodiments, the carbohydrates are provided by a corn syrup, a beet sugar, a cane sugar, a juice, or a tea. In another particular embodiment, the hydration is a flavanol that provides cellular rehydration. Non-limiting examples of suitable flavanols for use in particular embodiments of this invention include catechin, epicatechin, gallocatechin, epigallocatechin, epicatechin gallate, epigallocatechin 3-gallate, theaflavin, theaflavin 3-gallate, theaflavin 3′-gallate, theaflavin 3,3′ gallate, thearubigin or combinations thereof. In a particular embodiment, the hydration product is a glycerol solution to enhance exercise endurance.

Probiotics comprise microorganisms that benefit health when consumed in an effective amount. Probiotics may include, without limitation, bacteria, yeasts, and fungi. Examples of probiotics include, but are not limited to, bacteria of the genus Lactobacilli, Bifidobacteria, Streptococci, or combinations thereof. In particular embodiments of the invention, the at least one probiotic is chosen from the genus Lactobacilli. Lactobacilli (i.e., bacteria of the genus Lactobacillus, hereinafter “L.”). Non-limiting examples of species of Lactobacilli found in the human intestinal tract include L. acidophilus, L. casei, L. fermentum, L. saliva roes, L. brevis, L. leichmannii, L. plantarum, L. cellobiosus, L. reuteri, L. rhamnosus, L. GG, L. bulgaricus, and L. thermophilus. According to other particular embodiments of this invention, the probiotic is chosen from the genus Bifidobacteria. Non-limiting species of Bifidobacteria found in the human gastrointestinal tract include B. angulatum, B. animalis, B. asteroides, B. bifidum, B. boum, B. breve, B. catenulatum, B. choerinum, B. coryneforme, B. cuniculi, B. dentium, B. gallicum, B. gallinarum, B indicum, B. longum, B. magnum, B. merycicum, B. minimum, B. pseudocatenulatum, B. pseudolongum, B. psychraerophilum, B. pullorum, B. ruminantium, B. saeculare, B. scardovii, B. simiae, B. subtile, B. thermacidophilum, B. thermophilum, B. urinalis, and B. sp. According to other particular embodiments of this invention, the probiotic is chosen from the genus Streptococcus. Streptococcus thermophilus is a gram-positive facultative anaerobe. Other non-limiting probiotic species of this bacteria include Streptococcus salivarus and Streptococcus cremoris.

Prebiotics are compositions that promote the growth of beneficial bacteria in the intestines. Prebiotics include, without limitation, mucopolysaccharides, oligosaccharides, polysaccharides, amino acids, vitamins, nutrient precursors, proteins and combinations thereof. According to a particular embodiment, the prebiotic is chosen from dietary fibers, including, without limitation, polysaccharides and oligosaccharides. Non-limiting examples of oligosaccharides that are categorized as prebiotics in accordance with particular embodiments of this invention include fructooligosaccharides, inulins, isomalto-oligosaccharides, lactilol, lactosucrose, lactulose, pyrodextrins, soy oligosaccharides, transgalacto-oligosaccharides, and xylo-oligosaccharides. According to other particular embodiments, the prebiotic is an amino acid.

As used herein, “a weight management agent” includes an appetite suppressant and/or a thermogenesis agent. As used herein, the phrases “appetite suppressant”, “appetite satiation compositions”, “satiety agents”, and “satiety ingredients” are synonymous. The phrase “appetite suppressant” describes macronutrients, herbal extracts, exogenous hormones, anorectics, anorexigenics, pharmaceutical drugs, and combinations thereof, that when delivered in an effective amount, suppress, inhibit, reduce, or otherwise curtail a person's appetite. The phrase “thermogenesis agent” describes macronutrients, herbal extracts, exogenous hormones, anorectics, anorexigenics, pharmaceutical drugs, and combinations thereof, that when delivered in an effective amount, activate or otherwise enhance a person's thermogenesis or metabolism.

Suitable weight management agents include macronutrients selected from the group consisting of proteins, carbohydrates, dietary fats, and combinations thereof. Carbohydrates generally comprise sugars, starches, cellulose and gums that the body converts into glucose for energy. Non-limiting examples of carbohydrates include polydextrose; inulin; monosaccharide-derived polyols such as erythritol, mannitol, xylitol, and sorbitol; disaccharide-derived alcohols such as isomalt, lactitol, and maltitol; and hydrogenated starch hydrolysates. Carbohydrates are described in more detail herein below. Dietary fats are lipids comprising combinations of saturated and unsaturated fatty acids. Polyunsaturated fatty acids have been shown to have a greater satiating power than mono-unsaturated fatty acids. Accordingly, the dietary fats embodied herein desirably comprise poly-unsaturated fatty acids, non-limiting examples of which include triacylglycerols.

In a particular embodiment, the weight management agent is an herbal extract. Non-limiting examples of plants whose extracts have appetite suppressant properties include plants of the genus Hoodia, Trichocaulon, Caralluma, Stapelia, Orbea, Asclepias, and Camelia. Other embodiments include extracts derived from Gymnema sylvestre, Kola Nut, Citrus Auran tium, Yerba Mate, Griffonia simplicifolia, Guarana, myrrh, guggul Lipid, and black current seed oil. In a particular embodiment, the herbal extract is derived from a plant of the genus Hoodia, species of which include H. alstonii, H. currorii, H. dregei, H. flava, H. gordonii, H. jutatae, H. mossamedensis, H. officinalis, H. parviflorai, H. pedicellata, H. pifera, H. ruschii, and H. triebneri. Hoodia plants are stem succulents native to southern Africa.

In another particular embodiment, the herbal extract is derived from a plant of the genus Caralluma, species of which include C. indica, C. fimbriata, C. attenuate, C. tuberculata, C. edulis, C. adscendens, C. stalagmifera, C. umbellate, C. penicillata, C. russeliana, C. retrospicens, C. arabica, and C. lasiantha. Carralluma plants belong to the same Subfamily as Hoodia, Asclepiadaceae.

In another particular embodiment, the at least one herbal extract is derived from a plant of the genus Trichocaulon. Trichocaulon plants are succulents that generally are native to southern Africa, similar to Hoodia, and include the species T. piliferum and T. officinale. In another particular embodiment, the herbal extract is derived from a plant of the genus Stapelia or Orbea, species of which include S. gigantean and O. variegate, respectively. Both Stapelia and Orbea plants belong to the same Subfamily as Hoodia, Asclepiadaceae.

In another particular embodiment, the herbal extract is derived from a plant of the genus Asclepias. Asclepias plants also belong to the Asclepiadaceae family of plants. Non-limiting examples of Asclepias plants include A. incarnate, A. curassayica, A. syriaca, and A. tuberose. Not wishing to be bound by any theory, it is believed that the extracts comprise steroidal compounds, such as pregnane glycosides and pregnane aglycone, having appetite suppressant effects. In a particular embodiment, the weight management agent is an exogenous hormone having a weight management effect. Non-limiting examples of such hormones include CCK, peptide YY, ghrelin, bombesin and gastrin-releasing peptide (GRP), enterostatin, apolipoprotein A-IV, GLP-1, amylin, somastatin, and leptin.

In certain embodiments, the osteoporosis management agent is at least one calcium source, i.e. any compound containing calcium, including salt complexes, solubilized species, and other forms of calcium. Non-limiting examples of calcium sources include amino acid chelated calcium, calcium carbonate, calcium oxide, calcium hydroxide, calcium sulfate, calcium chloride, calcium phosphate, calcium hydrogen phosphate, calcium dihydrogen phosphate, calcium citrate, calcium malate, calcium citrate malate, calcium gluconate, calcium tartrate, calcium lactate, solubilized species thereof, and combinations thereof. According to a particular embodiment, the osteoporosis management agent is a magnesium source, i.e. any compound containing magnesium, including salt complexes, solubilized species, and other forms of magnesium. Non-limiting examples of magnesium sources include magnesium chloride, magnesium citrate, magnesium gluceptate, magnesium gluconate, magnesium lactate, magnesium hydroxide, magnesium picolate, magnesium sulfate, solubilized species thereof, and mixtures thereof. In another particular embodiment, the magnesium source comprises an amino acid chelated or creatine chelated magnesium. In other embodiments, the osteoporosis agent is chosen from vitamins D, C, K, their precursors and/or beta-carotene and combinations thereof. Numerous plants and plant extracts also have been identified as being effective in the prevention and treatment of osteoporosis. Not wishing to be bound by any theory, it is believed that the plants and plant extracts stimulates bone morphogenic proteins and/or inhibits bone resorption, thereby stimulating bone regeneration and strength. Non-limiting examples of suitable plants and plant extracts as osteoporosis management agents include species of the genus Taraxacum and Amelanchier, as disclosed in U.S. Patent Publication No. 2005/0106215, and species of the genus Lindera, Artemisia, Acorus, Carthamus, Carum, Cnidium, Curcuma, Cyperus, Juniperus, Prunus, Iris, Cichorium, Dodonaea, Epimedium, Erigonoum, Soya, Mentha, Ocimum, thymus, Tanacetum, Plantago, Spearmint, Bixa, Vitis, Rosemarinus, Rhus, and Anethum, as disclosed in U.S. Patent Publication No. 2005/0079232.

Examples of suitable phytoestrogens for embodiments of this invention include, but are not limited to, isoflavones, stilbenes, lignans, resorcyclic acid lactones, coumestans, coumestroI, equol, and combinations thereof. Isoflavones belong to the group of phytonutrients called polyphenols. In general, polyphenols (also known as “polyphenolics”), are a group of chemical substances found in plants, characterized by the presence of more than one phenol group per molecule. Suitable phytoestrogen isoflavones in accordance with embodiments of this invention include genistein, daidzein, glycitein, biochanin A, formononetin, their respective naturally occurring glycosides and glycoside conjugates, matairesinol, secoisolariciresinol, enterolactone, enterodiol, textured vegetable protein, and combinations thereof.

Long-chain primary aliphatic saturated alcohols are a diverse group of organic compounds. The term long-chain refers to the fact that the number of carbon atoms in these compounds is at least 8 carbons. Non-limiting examples of particular long-chain primary aliphatic saturated alcohols for use in particular embodiments of the invention include the 8 carbon atom 1-octanol, the 9 carbon 1-nonanol, the 10 carbon atom 1-decanol, the 12 carbon atom 1-dodecanol, the 14 carbon atom 1-tetradecanol, the 16 carbon atom 1-hexadecanol, the 18 carbon atom 1-octadecanol, the 20 carbon atom 1-eicosanol, the 22 carbon 1-docosanol, the 24 carbon 1-tetracosanol, the 26 carbon 1-hexacosanol, the 27 carbon 1-heptacosanol, the 28 carbon 1-octanosol, the 29 carbon 1-nonacosanol, the 30 carbon 1-triacontanol, the 32 carbon 1-dotriacontanol, and the 34 carbon 1-tetracontanol. In a particularly desirable embodiment of the invention, the long-chain primary aliphatic saturated alcohols are policosanol. Policosanol is the term for a mixture of long-chain primary aliphatic saturated alcohols composed primarily of 28 carbon 1-octanosol and 30 carbon 1-triacontanol, as well as other alcohols in lower concentrations such as 22 carbon 1-docosanol, 24 carbon 1-tetracosanol, 26 carbon 1-hexacosanol, 27 carbon 1-heptacosanol, 29 carbon 1-nonacosanol, 32 carbon 1-dotriacontanol, and 34 carbon 1-tetracontanol.

At least 44 naturally-occurring phytosterols have been discovered, and generally are derived from plants, such as corn, soy, wheat, and wood oils; however, they also may be produced synthetically to form compositions identical to those in nature or having properties similar to those of naturally-occurring phytosterols. According to particular embodiments of this invention, non-limiting examples of phytosterols well known to those or ordinary skill in the art include 4-desmethylsterols (e.g., β-sitosterol, campesterol, stigmasterol, brassicasterol, 22-dehydrobrassicasterol, and Δ5-avenasterol), 4-monomethyl sterols, and 4,4-dimethyl sterols (triterpene alcohols) (e.g., cycloartenol, 24-methylenecycloartanol, and cyclobranol).

According to particular embodiments of this invention, non-limiting examples of phytostanols include β-sitostanol, campestanol, cycloartanol, and saturated forms of other triterpene alcohols.

Both phytosterols and phytostanols, as used herein, include the various isomers such as the α and β isomers (e.g., α-sitosterol and β-sitostanol, which comprise one of the most effective phytosterols and phytostanols, respectively, for lowering serum cholesterol in mammals). The phytosterols and phytostanols of the present invention also may be in their ester form. Non-limiting examples of suitable phytosterol and phytostanol esters include sitosterol acetate, sitosterol oleate, stigmasterol oleate, and their corresponding phytostanol esters. The phytosterols and phytostanols of the present invention also may include their derivatives.

Generally, the amount of functional ingredient in the composition varies widely depending on the particular composition and the desired functional ingredient. Those of ordinary skill in the art will readily ascertain the appropriate amount of functional ingredient for each composition.

Consumables

Disclosed herein are consumables (e.g., beverages) comprising the sweetener compositions or flavor compositions disclosed herein.

Brazzein (or analog thereof) or the brazzein-containing sweetener compositions or flavor modifying compositions disclosed herein can be incorporated in any known edible material (referred to herein as a “consumable, a “sweetenable composition” or “flavor and/or taste modified composition”), such as, edible gel mixes and compositions, dental compositions, foodstuffs (confections, condiments, chewing gum, cereal compositions, baked goods, dairy products, and tabletop sweetener compositions) beverages and beverage products.

In one embodiment, the consumable is a beverage or beverage product. Put another way, disclosed herein is a beverage or beverage product comprising brazzein (or analog thereof) or the brazzein-containing sweetener compositions or flavor modifying compositions disclosed herein.

As used herein a “beverage product” is a ready-to-drink beverage, a beverage concentrate, a beverage syrup, or a powdered beverage. Suitable ready-to-drink beverages include carbonated and non-carbonated beverages. Carbonated beverages include, but are not limited to, cola, ginger-ale, soft drinks and root beer. Non-carbonated beverages include, but are not limited to fruit juice, fruit-flavored juice, vegetable juice, vegetable-flavored juice, sports drinks, energy drinks, plant protein beverage, near water drinks (e.g., water with natural or synthetic flavorants), tea type (e.g. black tea, green tea, red tea, oolong tea), coffee, cocoa drink, beverage containing milk components (e.g. milk beverages, coffee containing milk components, café au lait, milk tea, fruit milk beverages).

In particular embodiments, the beverage is a flavored black tea beverage, a zero calorie enhanced water beverage or an orange-flavored sparkling beverage.

Beverage concentrates and beverage syrups are prepared with an initial volume of liquid matrix (e.g. water) and the desired beverage ingredients. Full strength beverages are then prepared by adding further volumes of water. Powdered beverages are prepared by dry-mixing all of the beverage ingredients in the absence of a liquid matrix. Full strength beverages are then prepared by adding the full volume of water.

Beverages contain water as the liquid matrix, i.e. the basic ingredient in which the ingredients—including the sweetener or sweetener compositions—are dissolved. Water of beverage quality, such as, for example deionized water, distilled water, reverse osmosis water, carbon-treated water, purified water, demineralized water and combinations thereof, can be used. Additional suitable liquid matrices include, but are not limited to phosphoric acid, phosphate buffer, citric acid, citrate buffer and carbon-treated water.

In one embodiment, a beverage or beverage product is disclosed that contains brazzein or an analog thereof. In a particular embodiment, the beverage contains a brazzein analog that has the same or increased sweetness relative to a beverage containing the same amount of wild-type brazzein.

In one embodiment, the concentration of brazzein (or an analog thereof) in the beverage is at or above its sweetness recognition threshold concentration or flavor recognition threshold concentration. In a particular embodiment, the concentration of brazzein is at least about 1%, at least about 5%, at least about 10%, at least about 15%, at least about 20%, at least about 25%, at least about 30%, about least about 35%, at least about 40%, about least about 45%, at least about 50% or more above its sweetness or flavor recognition threshold concentration.

In one embodiment, the beverage or beverage product contains brazzein (or analog thereof) in an amount from about 1 ppm to about 50 ppm, such as, for example, from about 5 ppm to about 50 ppm, from about 5 ppm to about 40 ppm, from about 5 ppm to about 30 ppm, from about 5 ppm to about 20 ppm, from about 5 ppm to about 10 ppm, from about 10 ppm to about 50 ppm, from about 10 ppm to about 40 ppm, from about 10 ppm to about 30 ppm, from about 10 ppm to about 20 ppm, from about 20 ppm to about 50 ppm, from about 20 ppm to about 40 ppm, from about 20 ppm to about 30 ppm, from about 30 ppm to about 50 ppm, from about 30 ppm to about 40 ppm or from about 40 ppm to about 50 ppm.

In a more particular embodiment, the beverage or beverage product contains brazzein (or analog thereof) in an amount from about 10 ppm to about 25 ppm.

In another embodiment, the beverage contains brazzein (or an analog thereof) in an amount from about 1 ppm to less than about 15 ppm. In one embodiment, the beverage contains brazzein (or analog thereof) in an amount from about 1 ppm to about 5 ppm, about 5 ppm to about 10 ppm or about 10 ppm to about 15 ppm.

In another embodiment, the beverage contains brazzein (or analog thereof) in an amount greater than about 15 ppm. In one embodiment, the beverage contains brazzein (or an analog thereof) in an amount between about 15 ppm and about 20 ppm, about 20 ppm and about 25 ppm, about 25 ppm and about 30 ppm, about 30 ppm and about 35 ppm, about 35 ppm and about 40 ppm, about 40 ppm and about 50 ppm, or about 50 ppm or greater.

In one embodiment, the beverage contains brazzein (or analog thereof) in an amount of about 1 ppm, about 3 ppm, about 5 ppm, about 10 ppm, about 12 ppm, about 15 ppm, about 18 ppm, about 20 ppm, about 22 ppm, about 25 ppm, about 28 ppm, about 30 ppm, about 35 ppm, about 40 ppm, about 45 ppm, about 50 ppm, about 55 ppm, about 60 ppm, about 65 ppm, about 70 ppm or about 75 ppm or greater.

The beverage can further include at least one additional sweetener. Any of the sweeteners detailed herein can be used, including natural, non-natural, or synthetic sweeteners.

In a particular embodiment, the at least one addition sweetener is one or more steviol glycosides. In a particular embodiment, the steviol glycoside is Reb M. In another particular embodiment, the steviol glycoside is Reb A. In one embodiment, the beverage contains, in addition to brazzein (or analog thereof) a steviol glycoside blend, e.g., Reb M and Reb A.

The amount of the steviol glycoside (e.g., Reb M, Reb A) may vary. In one embodiment, the amount of the steviol glycoside provides less than about 8 sucrose equivalence (SE), e.g., about 7.5 SE, about 7.0 SE, about 6.5 SE or about 6.0 SE or less. In another embodiment, the amount of steviol glycoside provides greater than about 8 sucrose equivalence (SE), e.g., about 8.5 SE, about 9.0 SE, about 9.5 SE, about 10 SE, about 10.5 SE, about 11 SE or about 12 SE or more.

In another embodiment, the amount of stevoil glycoside (e.g., Reb M, Reb A) is less than about 450 ppm. In a particular embodiment, the amount of steviol glycoside is less than about 400 ppm, less than about 350 ppm, less than about 300 ppm, less than about 300 ppm, less than about 250 ppm, less than about 200 ppm, less than about 150 ppm or about 100 ppm or less.

In another embodiment, the amount of steviol glycoside (e.g., Reb M, Reb A) is between about 100 ppm and about 450 ppm, more particularly, between about 100 ppm and about 400 ppm, between about 100 ppm and about 350 ppm, between about 100 ppm and about 300 ppm, between about 100 ppm and about 250 ppm, between about 100 ppm and about 200 ppm, or between about 100 ppm and 250 ppm.

In a further embodiment, the amount of the stevoil glycoside (e.g., Reb M, Reb A) is between about 100 ppm and about 200 ppm, more particularly, about 110 ppm, about 120 ppm, about 130 ppm, about 140 ppm, about 150 ppm, about 160 ppm, about 170 ppm, about 180 ppm, about 190 ppm or about 200 ppm.

In a further embodiment, the amount of the stevoil glycoside (e.g., Reb M, Reb A) is between about 200 ppm and about 300 ppm, and more particularly, about 210 ppm, about 220 ppm, about 230 ppm, about 240 ppm, about 250 ppm, about 260 ppm, about 270 ppm, about 280 ppm, about 290 ppm, or about 300 ppm.

In another embodiment, the amount of the stevoil glycoside (e.g., Reb M, Reb A) is between about 300 ppm and about 400 ppm, and more particularly, about 310 ppm, about 320 ppm, about 330 ppm, about 340 ppm, about 350 ppm, about 360 ppm, about 370 ppm, about 380 ppm, about 390 ppm or about 400 ppm.

In another embodiment, the amount of the stevoil glycoside (e.g., Reb M, Reb A) is between about 400 ppm and about 500 ppm, and more particularly, about 410 ppm, about 420 ppm, about 430 ppm, about 440 ppm, about 450 ppm, about 460 ppm, about 470 ppm, about 480

In a particular embodiment, the beverage comprises brazzein (or analog thereof) in an amount between about 1 ppm and about 100 ppm and at least one steviol glycoside (e.g., Reb M, Reb A), wherein the amount of the at least one steviol glycoside is sufficient to provide less than about 8 SE, e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In another particular embodiment, the beverage comprises beverage comprises brazzein (or analog thereof) in an amount between about 1 ppm and about 100 ppm and at least one steviol glycoside (e.g., Reb M, Reb A), wherein the amount of the at least one steviol glycoside is about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In a particular embodiment, the beverage comprises brazzein (or analog thereof) in an amount between about 1 ppm and about 30 ppm, more particularly, about 1 ppm and about 15 ppm or about 15 ppm and about 30 ppm, and Reb M in an amount sufficient to provide less than about 8 SE, for example, e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In another particular embodiment, the beverage comprises brazzein (or analog thereof) in an amount between about 1 ppm and about 30 ppm, more particularly, between about 1 ppm and about 15 ppm or between about 15 ppm and about 30 ppm, and Reb M in an amount about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In another particular embodiment, a beverage comprises brazzein (or analog thereof) in an amount between about 1 ppm and about 30 ppm, more particularly, between about 1 ppm and about 15 ppm or between about 15 ppm and about 30 ppm, and Reb M in an amount about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

For example, a beverage comprises brazzein (or analog thereof) in an amount from about 1 ppm to about 30 ppm and RebM80 in an amount from about 200 ppm to about 400 ppm. In a more particular embodiment, a beverage comprises brazzein (or analog thereof) in an amount from about 20 ppm to about 30 ppm and RebM80 in an amount from about 300 to about 400 ppm. In a still more particular embodiment, a beverage comprises brazzein (or analog thereof) in an amount of about 25 ppm and RebM80 in an amount of about 315 ppm.

A beverage of the present invention comprises brazzein (or analog thereof) in an amount from about 1 ppm to about 50 ppm and RebM80 in an amount from about 200 ppm to about 400 ppm. In preferred embodiments, the beverage of the present invention tastes similar to a beverage sweetened with RebM or RebM80 only, wherein the beverage of the present invention and the RebM or RebM80-sweetened beverages have similar sucrose equivalence. In a more particular embodiment, a beverage having a citric acid matrix comprises brazzein (or analog thereof) in an amount from about 20 ppm to about 30 ppm and RebM80 in an amount from about 300 to about 400 ppm. In a still more particular embodiment, a beverage having a citric acid matrix comprises brazzein (or analog thereof) in an amount of about 25 ppm and RebM80 in an amount of about 315 ppm. In another more particular embodiment, a lemon-lime diet carbonated beverage comprises brazzein in an amount from about 30 ppm to about 50 ppm and RebM80 in an amount from about 150 ppm to about 350 ppm. In a still more particular embodiment, a lemon-lime diet carbonated beverage comprises brazzein in an amount of about 40 ppm and RebM80 in an amount of about 210 ppm.

In a particular embodiment, the beverage comprises brazzein (or analog thereof) in an amount between about 10 ppm and about 25 ppm and Reb M in an amount sufficient to provide less than about 8 SE, e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In another particular embodiment, the beverage comprises brazzein (or analog thereof) in an amount between about 10 ppm and about 25 ppm, and Reb M in an amount about 450 ppm or less, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In another particular embodiment, the beverage comprises brazzein (or analog thereof) in an amount between about 1 ppm and about 50 ppm and at least one additional sweetener (e.g., Reb M, Reb A, siamenoside I, mogroside IV), wherein the amount of the at least one additional sweetener is less than about 450 ppm, e.g., about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In a particular embodiment, the beverage comprises brazzein (or an analog thereof) in an amount between about 1 ppm and about 100 ppm and Reb A in an amount sufficient to provide less than about 8 SE., e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In a particular embodiment, the beverage comprises brazzein (or analog thereof) in an amount between about 1 ppm and about 30 ppm, more particularly between about 1 ppm and about 15 ppm or between about 15 ppm and about 30 ppm, Reb A in an amount less than about 450 ppm, e.g, about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

In a particular embodiment, the beverage comprises brazzein (or an analog thereof) in an amount between about 10 ppm and about 25 ppm and Reb A in an amount sufficient to provide less than about 8 SE., e.g., about 7.5 SE, about 7.0 SE, about 6.5 SE, about 6 SE, about 5.5 SE, about 5.0 SE, about 4.5 SE, about 4.0 SE, about 3.5 SE, about 3.0 SE, about 2.5 SE, about 2.0 SE, about 1.5 SE or about 1.0 SE.

In a particular embodiment, the beverage comprises brazzein (or analog thereof) in an amount between about 10 ppm and about 25 ppm, Reb A in an amount less than about 450 ppm, e.g, about 425 ppm, about 400 ppm, about 375 ppm, about 350 ppm, about 325 ppm, about 300 ppm, about 275 ppm, about 250 ppm, about 225 ppm, about 200 ppm, about 175 ppm, about 150 ppm, about 125 ppm or about 100 ppm or less.

The ppm ratio of brazzein (or analog thereof) and the one or more steviol glycosides in the beverage may vary. In one embodiment, the ppm ratio of brazzein (or analog thereof) and the one or more steviol glycosides is between about 1:2 and about 1:40, more particularly, between about 1:5 and about 1:35, even more particularly, between about 1:10 and about 1:25.

In a particular embodiment, the ppm ratio of brazzein (or analog thereof) and the one or more steviol glycosides is about 1:30 or less, more particularly, about 1:25.

In one embodiment, the steviol glycoside is Reb M and the ppm ratio of brazzein (or analog thereof) and Reb M is between about 1:10 and about 1:20, more particularly, about 1:10, about 1:12, about 1:14, about 1:16, about 1:18 or about 1:20.

In another embodiment, the steviol glycoside is Reb A and the ppm ratio of brazzein (or analog thereof) and Reb A is about 1:40 or less, more particularly, between about 1:20 and about 1:30, even more particularly, about 1:20, about 1:22, about 1:24, about 1:26, about 1:28 or about 1:30.

In another embodiment, the steviol glycoside is Reb A and the ppm ratio of brazzein (or analog thereof) and Reb A is about 1:70 or less, more particularly, less than about 1:60, less than about 1:50 or less than about 1:40. In a particular embodiment, the ppm ratio of brazzein (or analog thereof) and Reb A is about 1:35.

In another embodiment, the steviol glycoside is Reb A and the ppm ratio of brazzein (or analog thereof) and Reb A is about 1:15 or greater, more particularly, greater than about 1:17, about 1:18, about 1:19 or about 1:20.

In another embodiment, the steviol glycoside is Reb A and the ppm ratio of brazzein (or analog thereof) and Reb A is about 1:10 or greater, more particularly, greater than about 1:12, about 1:14 or about 1:16.

In one embodiment, the beverage comprises brazzein (or an analog thereof) and Reb M, wherein brazzein is present in an amount from about 15 ppm to about 30 ppm, more particularly, about 20 ppm to about 25 ppm, and Reb M (e.g., Reb M80) is present in an amount from about 300 ppm to about 350 ppm, more particularly, about 315 ppm to about 325 ppm (i.e. the Reb M provides about 8 SE). According to this embodiment, the ratio of brazzein to Reb M is between about 1:10 and about 1:20, more particularly about 1:0 1:12, about 1:14, about 1:16, about 1:18 or about 1:20. In one embodiment, the amount of Reb M provides about 8 sucrose equivalence (SE).

In one embodiment, the beverage comprises brazzein (or analog thereof) and Reb M, wherein brazzein is present in an amount from about 1 ppm to about 20 ppm, more particularly about 1 ppm to about 15 ppm, and Reb M is present in an amount of about 110 ppm to about 140 ppm, more particularly about 115 ppm, and even more particularly, about 120 ppm. In a particular embodiment, the ppm ratio of brazzein to Reb M is about 1:5 to about 1:10, more particularly about 1:7.

In one embodiment, the beverage comprises brazzein (or an analog thereof) and Reb M, wherein brazzein is present in an amount from about 1 ppm to about 15 ppm, more particularly, about 1 ppm to about 5 ppm, even more particularly, about 3 ppm, and Reb M is present in an amount between about 450 ppm and about 500 ppm, more particularly, about 470 ppm to about 475 ppm, even more particularly, about 472 ppm. In a particular embodiment, Reb M is present in an amount that provides greater than about 9 sucrose equivalence (SE).

In one embodiment, the beverage comprises brazzein (or an analog thereof) and Reb A, wherein brazzein is present in an amount from about 10 ppm to about 20 ppm, more particularly, about 15 ppm to about 20 ppm, and Reb A is present in an amount of about 375 ppm to about 425 ppm, more particularly, about 400 ppm. In one embodiment, the amount of Reb A provides about 7 SE. According to this embodiment, a ppm ratio of brazzein to Reb A between about 1:20 ppm and about 1:30 ppm ratio of brazzein to Reb A has at least one improved organoleptic property (e.g., taste) compared to a ppm ratio of brazzein to Reb A, more particularly about 1:20 ppm, about 1:22 ppm, about 1:24 ppm, about 1:26 ppm, about 1:28 ppm or about 1:30 ppm.

In one embodiment, the beverage comprises brazzein (or an analog thereof) and Reb A, wherein brazzein is present in an amount between about 5 ppm and about 15 ppm, more particular, about 10 ppm, and Reb A is present in an amount of between about 325 ppm and about 375 ppm, more particularly, about 350 ppm. In one embodiment, the amount of Reb A provides between about 6 and about 7 sucrose equivalence (SE). According to this embodiment, a ppm ratio of brazzein to Reb A of about 1:30 has at least one improved organoleptic property compared to a ppm ratio of brazzein to Reb A of about 1:70 or about 1:17.

In one embodiment, a beverage is provided that comprises brazzein and Reb A, wherein the amount of brazzein is between about 25 ppm and about 35 ppm, more particularly, about 30 ppm, and the amount of Reb A is between about 220 ppm and about 240 pm, more particularly, about 230 ppm. According to this embodiment, a ppm ratio of brazzein to Reb A of greater than about 1:10 has at least one improved organoleptic property than a ppm ratio of brazzein to Reb A of less than about 1:10 ppm. In a particular embodiment, a ppm ratio of brazzein to Reb A of about 1:12, about 1:14, about 1:16 or about 1:18 has at least one improved organoleptic property than a ppm ratio of brazzein of about 1:8, about 1:6, about 1:4, about 1:2 or about 1:1 ppm.

In one embodiment, a beverage is provided that comprises brazzein and Reb A, wherein the amount of brazzein is between about 50 ppm and about 70 ppm, more particularly, about 60 ppm, and the amount of Reb A is between about 100 ppm and about 130 ppm, more particularly, about 120 ppm. According to this embodiment, the ppm ratio of brazzein to Reb A is about 1:2.

In one embodiment, a beverage comprises from about 300 ppm to about 350 ppm Reb A and from about 10 ppm to about 50 ppm brazzein. In preferred embodiments, the beverage comprising Reb A and brazzein tastes better and/or has positive synergy compared to a Reb A-sweetened beverage, wherein the beverage comprising Reb A and brazzein and beverage sweetened with Reb A have the same sucrose equivalence.

In one embodiment, a beverage comprises from about 30 ppm to about 50 ppm brazzein, from about 300 ppm to about 450 ppm siamenoside I, and optionally from about 50 ppm to about 100 ppm RebM80. In preferred embodiments, the beverage tastes similar or better than either a siamenoside I-sweetened control or a RebM80-sweetened control, wherein the beverage of the present invention and the siamenoside I- and/or RebM80-sweetened control beverages have the same sucrose equivalence. In a particular embodiment, a beverage comprises from about 40 ppm to about 50 ppm brazzein and from about 350 ppm to about 450 ppm siamenoside I, with no RebM80. In a more particular embodiment, the beverage comprises from about 40 ppm to about 45 ppm brazzein and from about 350 ppm to about 450 ppm siamenoside I, with no RebM80. In a still more particular embodiment, the beverage comprises about 45 ppm brazzein and about 410 ppm siamenoside I, with no RebM80. In another embodiment, a beverage comprises from about 25 to about 30 ppm brazzein, from about 300 ppm to about 400 ppm siamenoside I and from about 50 ppm to about 100 ppm RebM80. In a more particular embodiment, a beverage comprises about 30 ppm brazzein, about 350 ppm siamenoside I and about 70 ppm RebM80.

In one embodiment, a beverage comprises from about 30 to about 50 ppm brazzein and from about 7 Brix to about 10 Brix high fructose corn syrup (HFCS). In preferred embodiments, the beverage of the present invention tastes similar or better than a HFCS-sweetened control (10 Brix). In a more particular embodiment, the beverage comprises from about 35 ppm to about 45 ppm brazzein and about 7 Brix to about 9 Brix HFCS. In a still more particular embodiment, the beverage comprises about 40 ppm brazzein and about 8 Brix HFCS.

In one embodiment, a reduced calorie beverage comprises from about 5 ppm to about 20 ppm brazzein and from about 100 ppm to about 200 ppm RebM. In preferred embodiments, the beverage of the present invention has a taste profile similar to a sucrose-sweetened control (10 Brix). In a particular embodiment, a reduced calorie still (non-carbonated) beverage comprises from about 10 ppm to about 15 ppm brazzein and from about 100 ppm to about 150 ppm RebM. In a still more particular embodiment, a reduced calorie still beverage comprises from about 10 ppm to about 15 ppm brazzein and about 115 ppm RebM. In another particular embodiment, a reduced calorie carbonated beverage comprises about 20 ppm brazzein and from about 100 to about 150 ppm RebM.

In one embodiment, a beverage comprises from about 5 ppm to about 20 ppm brazzein and from about 300 ppm to about 500 ppm A95. In a particular embodiment, the beverage comprises from about 15 ppm to about 20 ppm brazzein and about 300 ppm to about 500 ppm A95. In a further embodiment, the beverage comprises about 15 ppm or about 20 ppm brazzein and about 400 ppm A95.

In one embodiment, the temporal profile, flavor profile and/or taste profile of the beverage is improved relative to a beverage that either does not contain brazzein or contains brazzein in an amount less than from about 20 ppm, even more particularly, less than about 15 ppm. In a particular embodiment, the temporal profile, flavor profile and/or taste profile of the beverage is more sugar-like. In one embodiment, the beverage has reduced sweetness linger, reduced bitterness, reduced bitter aftertaste, reduced astringency, improved mouthfeel (e.g., greater fullness or body) or the like.

The beverages can further include additives including, but are not limited to, carbohydrates, polyols, amino acids and their corresponding salts, poly-amino acids and their corresponding salts, sugar acids and their corresponding salts, nucleotides, organic acids, inorganic acids, organic salts including organic acid salts and organic base salts, inorganic salts, bitter compounds, flavorants and flavoring ingredients, astringent compounds, proteins or protein hydrolysates, surfactants, emulsifiers, flavonoids, alcohols, polymers and combinations thereof. Any suitable additive described herein can be used.

The beverage can further contain one or more functional ingredients, detailed above. Functional ingredients include, but are not limited to, vitamins, minerals, antioxidants, preservatives, glucosamine, polyphenols and combinations thereof. Any suitable functional ingredient described herein can be used.

It is contemplated that the pH of the sweetened composition, such as, for example, a beverage, does not materially or adversely affect the taste of the sweetener. A non-limiting example of the pH range of the sweetenable composition may be from about 1.8 to about 8. A further example includes a pH range from about 2 to about 5. In a particular embodiment, the pH of a beverage is from about 3 to about 3.25.

The temperature of a beverage comprising brazzein (or analogs thereof) may, for example, range from about 4° C. to about 100° C., such as, for example, from about 4° C. to about 25° C.

The beverage can be a high-calorie beverage that has about 120 calories per 8 oz serving.

The beverage can be a mid-calorie beverage that has about 80 calories per 8 oz serving.

The beverage can be a low-calorie beverage that has less than 40 calories per 8 oz serving.

The beverage can be a zero-calorie that has less than 5 calories per 8 oz. serving.

In one embodiment, the beverage or beverage product has a low glycemic index. The glycemic index is a value assigned to foods based on how slowly or how quickly those foods cause increases in blood glucose level. Low glycemic index diets (including but not limited to beverages) are intended to achieve a more beneficial effect on blood glucose control in people with diabetes mellitus and may also provide metabolic benefits for the general population.

In a particular embodiment, the beverage or beverage product has a glycemic index that is at least 10% lower than the glycemic index of a substantially similar product made using conventional sweeteners (e.g., sucrose-based sweeteners). Methods for testing the glycemic index of a beverage have been described, e.g., as provided in Wolever, et al. Nutrition Research 23:621-629, 2003. In a particular embodiment, the beverage of beverage product has a glycemic index (GI) of about 55 or less. In a particular embodiment, the beverage or beverage product is a nutritional beverage with a low glycemic index.

In one embodiment, a beverage is disclosed that comprises between about 1 and about 30 ppm and less than about 500 ppm of one or more steviol glycosides, wherein the liquid matrix of the beverage is selected from the group consisting of water, phosphoric acid, phosphate buffer, citric acid, citrate buffer, carbon-treated water and combinations thereof. The pH of the beverage can be from about 3 to about 3.5. The beverage can further include additives, such as, for example, erythritol. The beverage can further include functional ingredients, such as, for example vitamins.

Methods for Improving Temporal or Flavor Profile

A method is disclosed for imparting a more sugar-like temporal profile, flavor profile and/or taste profile to a consumable (e.g. a beverage), comprising adding brazzein (or an analog thereof) or the brazzein-containing sweetener compositions or flavor modifying compositions disclosed herein to the consumable. The consumable may be referred to as a sweetenable composition or a flavor modifiable composition.

Without being bound by any particular theory, it is believed that the brazzein may bind to mucins in the oral cavity. Mucins are the principal organic constituents of mucus, the slimy visco-elastic material that coats all mucosal surfaces. Structurally, mucins are high-molecular weight epithelial glycoproteins with a high content of clustered oligosaccharides O-glycosidically linked to tandem repeat peptides rich in threonine, serine, and proline.

Within the mouth, muscins coat the hard and soft tissues. Several salivary mucins are known, including MUC5B, MUC7 (previously known as MG2), MUC19, MUC1, and MUC4. Based on macromolecular characteristics, they can be classified into high (>1000 kDa) and low (200-300 kDa) molecular weight forms. Salivary mucins are synthesized by the mucus acinar cells of the paired submandibular (SMG) and sublingual (SLG) glands, as well as minor salivary glands distributed throughout the palatal and buccal mucosa.

The method can further include the addition of other sweeteners, additives, functional ingredients and combinations thereof. Any sweetener, additive or functional ingredient detailed herein can be used.

In one embodiment, the method or imparting a more sugar-like temporal profile, flavor profile to a consumable (e.g., a beverage) comprises combining adding brazzein (or analog thereof) or the brazzein-containing sweetener composition or flavor modifying composition to the consumable, thereby imparting a more sugar-like temporal profile or flavor profile.

As used herein, the “sugar-like” characteristics include any characteristic similar to that of sucrose and include, but are not limited to, maximal response, flavor profile, temporal profile, taste profile, adaptation behavior, mouthfeel, concentration/response function, tastant/and flavor/sweet taste interactions, spatial pattern selectivity, and temperature effects.

These characteristics are dimensions in which the taste of sucrose is different from the tastes of brazzein (or analog thereof). Of these, however, the flavor profile and temporal profile are particularly important. In a single tasting of a sweet food or beverage, differences (1) in the attributes that constitute a sweetener's flavor profile and (2) in the rates of sweetness onset and dissipation, which constitute a sweetener's temporal profile, between those observed for sucrose and for brazzein (or analog thereof) can be noted.

In on embodiment, the method disclosed herein improves one or more organoleptic property of the consumable compared to a consumable that does not contain the sweetener composition or flavor modifying composition disclosed herein. In certain embodiments, the improvements is measured by a sensory test. The sensory test may be a taste test, a blind test, or a combination thereof. A sensory test can use one or more various protocols. For example, a sensory test can be the “triangle method”, follow ISO requirements, or a combination thereof. The taste test can be the average of multiple trials.

A taste test may be a screening test, a professional taste test, or a market research test. A screening test may be performed by at least 1, 2, 3, 4, 5, 6, 7, 8, or 9 taste testers. A professional taste test may be performed by at least 10, 15, 20, 25, or 30 taste testers. A market research test may be performed by at least 31, 40, 50, 60, 70, 80, 90, 100, 150, 200, 300, 400, or 500 taste testers. In some cases, a taste tester can be a person with average taste perception, a professional taste tester, a person who has passed a tasting exam by correctly identifying foods or food components, or a person who can identify the relative amounts of a taste or flavor (e.g., correctly sequence varying amounts of sugar in water).

In one embodiment, whether or not a characteristic is more sugar-like is determined by an expert sensory panel who taste compositions comprising sugar and compositions comprising brazzein (or analog thereof), optionally in combination with at least one steviol (e.g., Reb M, Reb A) or HFCS, both with and without additives and provide their impression as to the similarities of the characteristics of the sweetener compositions, both with and without additives, with those comprising sugar. A suitable procedure for determining whether a composition has a more sugar-like taste is described in embodiments described herein below.

In a particular embodiment, a panel of assessors is used to measure the reduction of sweetness linger. Briefly described, a panel of assessors (generally 8 to 12 individuals) is trained to evaluate sweetness perception and measure sweetness at several time points from when the sample is initially taken into the mouth until 3 minutes after it has been expectorated. Using statistical analysis, the results are compared between samples containing additives and samples that do not contain additives. A decrease in score for a time point measured after the sample has cleared the mouth indicates there has been a reduction in sweetness perception.

The panel of assessors may be trained using procedures well known to those of ordinary skill in the art. In a particular embodiment, the panel of assessors may be trained using the Spectrum™ Descriptive Analysis Method (Melgaard et al, Sensory Evaluation Techniques, 3^{rd} edition, Chapter 11). Desirably, the focus of training should be the recognition of and the measure of the basic tastes; specifically, sweet. In order to ensure accuracy and reproducibility of results, each assessor should repeat the measure of the reduction of sweetness linger about three to about five times per sample, taking at least a five-minute break between each repetition and/or sample and rinsing well with water to clear the mouth.

Generally, the method of measuring sweetness comprises taking a 10 mL sample into the mouth, holding the sample in the mouth for 5 seconds and gently swirling the sample in the mouth, rating the sweetness intensity perceived at 5 seconds, expectorating the sample (without swallowing following expectorating the sample), rinsing with one mouthful of water (e.g., vigorously moving water in mouth as if with mouth wash) and expectorating the rinse water, rating the sweetness intensity perceived immediately upon expectorating the rinse water, waiting 45 seconds and, while waiting those 45 seconds, identifying the time of maximum perceived sweetness intensity and rating the sweetness intensity at that time (moving the mouth normally and swallowing as needed), rating the sweetness intensity after another 10 seconds, rating the sweetness intensity after another 60 seconds (cumulative 120 seconds after rinse), and rating the sweetness intensity after still another 60 seconds (cumulative 180 seconds after rinse). Between samples take a 5-minute break, rinsing well with water to clear the mouth.

In some embodiments, the temporal profile, flavor profile and/or taste profile is evaluated in vitro, e.g., in a suitable assay such an in vitro system based on a cellular model overexpressing sweet and/or bitter receptor. In a particular embodiment, the in vitro system comprises TAS1R2/TAS1R3 receptors.

In one embodiment, the sweetener composition or flavor and/or taste modifying composition disclosed herein is capable of modifying one or more properties of the consumable including but not limited to mouthfeel, bitterness, bitter aftertaste or sweetness linger.

Mouthfeel refers to the textural aspects of a food or beverage responsible for producing characteristic tactile sensations perceived at the lining of the mouth, including the tongue, gums and teeth. These may include, but are not limited to, astringency, viscosity, slipperiness and mouth-coating. Mouthfeel is a fundamental sensory attribute which, along with taste and smell, determines the overall flavor of a consumable.

In one embodiment, brazzein (or analog thereof) or the brazzein-containing sweetener composition and/or flavor modifying composition disclosed herein imparts an improved mouthfeel to a consumable (e.g., beverage) to which it is added relative to a conventional sweetener composition or flavor and/or taste modifying composition. In one embodiment, the “improved mouthfeel” can be determined by a taste panel consuming said beverage in comparison to the same beverage without the taste modifying composition or an active component thereof. In a particular embodiment, the mouthfeel is improved by about 5%, about 10%, about 15%, about 20% or about 25% or more. In a particular embodiment the improvement mouthfeel is characterized as increased fullness, body or richness. In a particular embodiment, the improved mouthfeel is characterized as a more syrup-y feel.

In another embodiment, the sweetener composition or flavor modifying composition disclosed herein improves (reduces) bitterness in a consumable (e.g., a beverage) to which it is added, relative to a conventional sweetener composition or flavor and/or taste modifying composition. This “improved bitterness” or “reduced bitterness” can be determined by a taste panel consuming said beverage in comparison to the same beverage without the taste modifying composition or an active component thereof. In a particular embodiment, the bitterness is reduced by about 5%, about 10%, about 15%, about 20% or about 25% or more.

In another embodiment, the sweetener composition or flavor modifying composition disclosed herein reduces the bitter aftertaste in a consumable (e.g., a beverage) to which it is added, relative to a conventional sweetener composition or flavor and/or taste modifying composition. This “reduced bitter aftertaste” can be determined by a taste panel consuming said beverage in comparison to the same beverage without the taste modifying composition or an active component thereof. In a particular embodiment, the bitter aftertaste is reduced by about 5%, about 10%, about 15%, about 20% or about 25% or more.

In a particular embodiment, the sweetener composition or flavor modifying composition disclosed herein reduces the bitter taste of consumable (e.g., a beverage) to which it is added where the reduction is measured by the Metachronic VAS Score Profile for Bitterness and more particularly, the reduction is at least about 0.5 points, about 1 point, about 1.5 points, about 2.0 points, about 2.5 points, about 3.0 points, about 3.5 points or about 4.0 points or more. In certain embodiments, the reduction is between about 0.5 and about 4.0 points, about 1.0 and about 3.5 points or about 1.5 and about 3.0 points.

In a further embodiment, the sweetener composition and/or flavor modifying composition disclosed herein has an improved (reduced) sweetness linger. In one embodiment, this “improved sweetness linger” can be examined by a taste panel consuming said beverage in comparison to the same beverage without the taste modifying composition or an active component thereof. In a particular embodiment, the sweetness linger is reduced by about 5%, about 10% about 15%, about 20% or about 25% or more.

In a further embodiment, flavor modifying composition disclosed herein provides a reduction in sourness. In one embodiment, this “reduced sourness” can be examined by a taste panel consuming said beverage in comparison to the same beverage without the taste modifying composition or an active component thereof. In a particular embodiment, the sourness is reduced by about 5%, about 10% about 15%, about 20% or about 25% or more.

### EXAMPLES

### Example 1: Brazzein+Reb M (Sweetener Composition)

Brazzein and RebM80 were added to a citric acid solution and a Lemon/Lime diet soda, respectively. The results are shown below in Table I and Table II, respectively.

TABLE I
In Citric Acid Solution

| Concentration | Sensory |
|---|---|
| 315 ppm RebM80 + 25 ppm | Sweeter than RebM only @472 ppm, |
| Brazzein | sweet profile is more sugar-like. |
| 472 ppm RebM80 (control) | |

TABLE II
In Lemon/Lime carbonated beverage

| Concentration | Sensory |
|---|---|
| 472 ppm RebM80 (control) | |
| 210 ppm RebM 80 + 40 ppm Brazzein | Similar to control |

### Example 2: Reb A Compared to Reb A+Brazzein (Sweetener Composition)

Reb A as compared to Reb A+Brazzein in citric acid solutions as described. The results are shown in Tables III and IV, below.

TABLE III
Citric Sucrose Control from 5-10 Brix

| | |
|---|---|
| 180 ppm RebA (Control) | Around 5-6 Brix SE, very barely sweet |
| 120 ppm RebA + 60 ppm | 8-10 Brix SE, sweet linger, delayed sweetness |
| Brazzein | onset, not very bitter, mouth coating, preferred |
| | to 180 ppm RebA control |

TABLE IV
In citric acid solution:

| | |
|---|---|
| 350 ppm RebA (Control) | metallic, very bitter, sour |
| 340 ppm RebA + 10 ppm | not much different than 350 ppm RebA |
| Brazzein | control, less metallic and bitter than |
| | control |
| 330 ppm RebA + 20 ppm | delayed onset, slightly sweeter, better |
| Brazzein | mouthfeel, sweetness linger |
| 320 ppm RebA + 30 ppm | better balance of sweet and sour than 20 |
| Brazzein | ppm and 10 ppm Brazzein replacement, much |
| | better mouthfeel than control, sweetness |
| | linger |
| 310 ppm RebA + 40 ppm | too sour upfront |
| Brazzein | |
| 300 ppm RebA + 50 ppm | more rounded sweetness than other samples |
| Brazzein | |

The results suggest that a Reb A+Brazzein blend has positive synergy compared with Reb A alone.

### Example 3: Siamenoside I+Brazzein (Sweetener Composition)

Brazzein, siamenoside I and/or RebM80 were added to a citric acid mock beverage. The results are shown Table V, below.

TABLE V

| | | Siamenoside | | |
|---|---|---|---|---|
| | Brazzein | I | RebM80 | Results |
| Control | N/A | N/A | 472 ppm | reference |
| Control | N/A | 600 ppm | N/A | reference |
| Test 4 | 40 ppm | 410 ppm | N/A | close but still slight |
| | | | | less sweet |
| Test 5 | 45 ppm | 410 ppm | N/A | close to 472 ppm RebM |
| Test 7 | 30 ppm | 350 ppm | 70 ppm | close but still slight |
| | | | | less sweet |

The result suggests that a siamenoside I blend with Brazzein and/or Reb M has positive synergy compared to RebM80 alone (sweetness quality).

### Example 4: Brazzein+HFCS (Sweetener Composition)

Brazzein and high fructose corn syrup (HFCS) were added to a citric acid mock beverage. The results are shown in Table V below:

TABLE V
In a citric acid mock beverage

| | Brazzein | HFCS | Results |
|---|---|---|---|
| Control | N/A | 10 Brix | reference |
| Test 3 | 30 ppm | 8 Brix | Similar but still lacking some |
| | | | sweetness |
| Test 4 | 40 ppm | 8 Brix | Similar to control |

HFCS blend with Brazzein provided similar taste as HFCS alone (sweetness quality).

### Example 5: Brazzein+Reb M (Taste Modifying Composition)

Reb M and brazzein were added to various beverages, as described below. A full sugar sample was provided as the control (10 Brix).

Reduced sugar 50% in still lemon/lime beverage

| Sample | 1 | 2 | 3 | 4 | 5 |
|---|---|---|---|---|---|
| RebM | 115 ppm | 0 | 115 | 115 | 115 |
| Brazzein | 0 | 63 ppm | 5 | 10 | 15 |
| Comments | Nice sweet | Slightly slow | Less sweet | Close to | Very |
| | intensity, thin on | on set, sour | and less | full sugar | close to |
| | mouth feel, sweet | upfront. Sweet | sugar like | control | full sugar. |
| | linger | lingering. | in taste. | | |

Reduced sugar 50% in carbonated lemon/lime beverage

| Sample | 1 | 2 | 3 | 4 |
|---|---|---|---|---|
| RebM | 115 | 115 | 115 | 140 |
| Brazzein | 10 | 15 | 20 | 20 |
| Comments | Less | Less sweet. | Closer to | Very |
| | sweet | The after | control. But | close to |
| | | taste | still slow | sugar. |
| | | sweetness | in onset. | |
| | | is close. | | |

### Example 6: Brazzein+Reb M (Taste Modifying Composition)

| Sample Name | Comments |
|---|---|
| 472 ppm RebM80 as | Very sweet, spike on sweetness, |
| Control (citric base) | sweetness lingering, bitter, thinner in |
| | mouthfeel |
| 472 ppm RebM80 + | More sour than control, sweeter than |
| 3 ppm Brazzein | control, syrupy mouthfeel, broader and |
| (citric base) | richer than control, more sugar like, |
| | not that different from Control, most |
| | similar to Control, less linger, not a |
| | big difference between 3 ppm Brazzein |
| | and 5 ppm Brazzein, better finish than |
| | Control |
| 472 ppm RebM80 + | More sour than control at the beginning, |
| 5 ppm Brazzein | sweeter than control, syrupy mouthfeel, |
| (citric base) | broader and richer than control, more |
| | sugar like |
| 472 ppm RebM80 + | Seemed more sour than Control and 3 ppm |
| 10 ppm Brazzein | and 5 ppm Brazzein added, less linger and |
| (citric base) | less bitter. |
| 472 ppm RebM80 + | Slightly less sweet than control though |
| 15 ppm Brazzein | very similar. Cleaner in after taste than |
| (citric base) | control. sweeter than 315 ppm RebM80 + |
| | 25 ppm Brazzein, sugary and sweet, same |
| | sweetness as Control, sweet lingering, sweet |
| | profile better than 3 ppm and 5 ppm |

Brazzein (1-15 ppm) improves the taste property in 472 ppm of RebM80 such as, sour, less sweet, cover the bitterness and lingering. More mouthfeel, richness and fullbody.

### Example 7: Brazzein+Reb A95 (Taste Modifying Composition)

“A95” refers to a steviol glycoside blend comprising primarily reb D and reb M. Methods of obtaining A95 are provided in WO 2017/059414, incorporated herein by reference. An exemplary A95 blend is the following:

| | Percent, as determined |
|---|---|
| A95 Component | by HPLC |
| Rebaudioside E | 0.86 |
| Rebaudioside O | 1.37 |
| Rebaudioside D | 63.95 |
| Rebaudioside N | 2.95 |
| Rebaudioside M | 25.37 |
| Rebaudioside A | 1.32 |
| Stevioside | 0.03 |
| Rebaudioside C | 0.01 |
| Rebaudioside B | 0.22 |
| Total Steviol Glycoside Content | 96.07 |

| Sample Name | Comments |
|---|---|
| 400 ppm A95 as | Very bitter, very harsh, sweet but more sour, |
| Control (citric base) | metallic |
| 400 A95 + 5 ppm | Not much sweeter than control, improvement |
| Brazzein (citric base) | from control, less bitter, less linger |
| 400 ppm A95 + | Sweeter than control, better mouthfeel, sweet |
| 10 ppm Brazzein | linger, less bitter, cuts linger from Reb A, much |
| (citric base) | better than 400 Reb A + 5 ppm Brazzein |
| 400 ppm A95 + | No bitterness, 10 ppm Brazzein and 15 ppm |
| 15 ppm Brazzein | Brazzein are very close, good mouthfeel, liked |
| (citric base) | 15 ppm Brazzein the best |
| 400 ppm A95 + | Sweet linger, sugary finish, sugar like |
| 20 ppm Brazzein | mouthfeel, good balance of the acid and |
| (citric base) | sweetness |

Brazzein (5 ppm to 20 ppm) can improve the mouthfeel, block bitterness and lingering in 400 ppm of A95.

1. A sweetener composition comprising (i) brazzein or an analog thereof and (ii) at least one additional sweetener selected from a steviol glycoside sweetener and a mogroside sweetener.

2. The sweetener composition of claim 1 wherein the steviol glycoside sweetener is selected from Rebaudioside M (“Reb M”), RebM80, Rebaudioside D (“Reb D”), A95 and Rebaudioside A (“Reb A”).

3. The sweetener composition of claim 1, wherein the mogroside sweetener is selected from siamenoside I and mogroside V.

4. A sweetener composition comprising brazzein and high fructose corn syrup (HFCS).

5. A beverage or beverage product comprising the sweetener composition of claim 1.

6. The beverage or beverage product of claim 5, wherein the brazzein is present in an amount between about 1 ppm and about 50 ppm.

7. The beverage or beverage product of claim 5, wherein the brazzein is present in an amount between about 1 ppm and about 40 ppm.

8. The beverage or beverage product of claim 5, wherein the brazzein is present in an amount selected from an amount between about 1 ppm and about 30 ppm, about 1 ppm and about 25 ppm, about 1 ppm and about 20 ppm or about 1 ppm and about 15 ppm.

9. The beverage or beverage product of claim 6, wherein the beverage or beverage product has at least one improved organoleptic property compared to a beverage or beverage product that does not contain the sweetener composition or flavor modifying composition, wherein the organoleptic property is selected from the group consisting of temporal profile, flavor profile, taste, bitter aftertaste, sweetness linger, mouthfeel or a combination thereof.

10. The beverage or beverage product of claim 9, wherein the at least one improved organoleptic property is a reduction in bitter aftertaste or an improvement in mouthfeel.

11. The beverage or beverage product of claim 9, wherein the at least one improved organoleptic property is an improvement in mouthfeel and the amount of brazzein is between about 1 ppm and about 40 ppm.

12. The beverage or beverage product of claim 6, wherein the beverage or beverage product is selected from a low-calorie or no-calorie beverage or beverage product.

13. The beverage or beverage product of claim 6, wherein the beverage is selected from the group consisting of cola, ginger-ale, soft drinks, root beer, fruit juice, fruit-flavored juice, vegetable juice, vegetable-flavored juice, sports drinks, energy drinks, plant protein beverage, near water drinks (e.g., water with natural or synthetic flavorants), tea type (e.g. black tea, green tea, red tea, oolong tea), coffee, cocoa drink, beverage containing milk components (e.g. milk beverages, coffee containing milk components, café au lait, milk tea, fruit milk beverages).

14. A method of improving at least one organoleptic property of a beverage or beverage product, comprising adding a sweetener composition of claim 1 to a liquid matrix thereby providing a beverage or beverage product having at least one improved organoleptic property.

15. The method of claim 14, wherein the steviol glycoside sweetener is selected from Rebaudioside M (“Reb M”), RebM80, Rebaudioside D (“Reb D”), A95 and Rebaudioside A (“Reb A”).

16. The method of claim 14, wherein the improved organoleptic property is selected from the group consisting of temporal profile, flavor profile, taste, bitter aftertaste, sweetness linger, mouthfeel or a combination thereof.

17. The method of claim 16, wherein the improved organoleptic property is a reduction in bitter aftertaste or an improvement in mouthfeel.

18. The method of claim 16, wherein the improved organoleptic property is an improvement in mouthfeel and the amount of brazzein present in the beverage or beverage product is an amount between about 1 ppm and about 40 ppm.

19. The method of claim 14, wherein brazzein is present in the beverage or beverage product in an amount between about 1 ppm and about 50 ppm.

20. The method of claim 14, wherein the brazzein is present in the beverage or beverage product in an amount selected from an amount between about 1 ppm and about 40 ppm, about 1 ppm and about 30 ppm, about 1 ppm and about 25 ppm, about 1 ppm and about 20 ppm or about 1 ppm and about 15 ppm.

21. The sweetener composition of claim 1, wherein the brazzein analog differs from the wild-type brazzein by at least one amino acid.

22. The sweetener composition of claim 21, wherein the brazzein analog has one of the at least one of the following: Asp40Lys, Glu41Ala, Lys42Ala, Asp50Lys, Tyr54Trp, Asp29Ala, Asp29Asn/Glu41Lys and Asp29Lys/Glu41Lys.

23. The sweetener composition of claim 1, wherein the brazzein has the amino acid sequence of SEQ ID NO: 1.

24. The sweetener composition of claim 1, wherein the brazzein is pGlu-brazzein or des-pGlu1-brazzein.

25. The sweetener composition of claim 1, wherein the mogroside sweetener is Mogroside V.

26. The sweetener composition of claim 2, wherein the Reb M has a purity of greater than about 95%.

27. The beverage or beverage product of claim 5, further comprising at least one organic acid additive salt that is a sodium, calcium, potassium, or magnesium salt of an organic acid.

28. The beverage or beverage product of claim 27, wherein the organic acid is selected from the group consisting of citric acid, malic acid, tartaric acid, fumaric acid, lactic acid, alginic acid, ascorbic acid, benzoic acid and adipic acid.

29. The method of claim 14, wherein the mogroside sweetener is selected from siamenoside I and mogroside V.

30. The method of claim 14, wherein the brazzein analog that differs from the wild-type brazzein by at least one amino acid.

31. The method of claim 30, wherein the brazzein analog has one of the at least one of the following: Asp40Lys, Glu41Ala, Lys42Ala, Asp50Lys, Tyr54Trp, Asp29Ala, Asp29Asn/Glu41Lys and Asp29Lys/Glu41Lys.

32. The method of claim 14, wherein the brazzein has the amino acid sequence of SEQ ID NO: 1.

33. The method of claim 14, wherein the brazzein is pGlu-brazzein or des-pGlu1-brazzein.
